(19) United States
(12) Patent Application Publication
(10) Pub. No.: US 2024/0165362 A1
(43) Pub. Date: May 23, 2024

(54) RESPIRATORY DEVICE CONNECTOR WITH LUMEN

(71) Applicant: Fisher & Paykel Healthcare Limited, Auckland (NZ)

(72) Inventors: Thomas Heinrich BARNES, Auckland (NZ); Xavier Roy Wallace SANDER, Auckland (NZ); Craig Karl WHITE, Auckland (NZ); Karan DEVA, Auckland (NZ); Alicia Jerram Hunter EVANS, Auckland (NZ); Anthony Behram ZEMKE, Auckland (NZ); Matthew Jon PAYTON, Auckland (NZ)

(21) Appl. No.: 18/551,171

(22) PCT Filed: Apr. 29, 2022
(86) PCT No.: PCT/IB2022/053980

§ 371 Date: Sep. 18, 2023

Related U.S. Application Data
(60) Provisional application No. 63/202,737, filed on Jun. 22, 2021.
(60) Provisional application No. 63/201,444, filed on Apr. 29, 2021.

(30) Foreign Application Priority Data
Jun. 22, 2021 (AU) 2021204223

(51) Int. Cl. A61M 16/08

(52) U.S. Cl. CPC A61M 16/085; A61M 16/0858; A61M 16/0875; A61M 2205/3344

(57) ABSTRACT

A connector is provided for coupling with an invasive patient interface for providing respiratory support to a patient. The connector comprises a connector body having: (i) an inlet port couplable with a flow source providing a flow of respiratory gas; (ii) a gases exit port; and (iii) a device port couplable with the invasive patient interface. The connector also has a lumen having a first end and a second end. The connector body defines a gas flow path between the inlet port and both the device port and the lumen first end and the lumen second end is disposed outside the device port. The lumen provides improved CO2 clearance. Some embodiments provide for gas sampling and/or variable expiratory resistance.

### TECHNICAL FIELD

The present disclosure relates to devices and systems delivering respiratory support to a patient. It relates particularly, but not exclusively, to connectors for use with invasive patient interfaces providing a flow of gas into a patient's airway, and to systems, methods and kits involving the same.

### BACKGROUND OF INVENTION

Patients usually require a form of respiratory support during medical procedures, particularly medical procedures which involve sedation or anaesthesia. A patient can be spontaneously breathing or apnoeic (which includes non-spontaneously breathing patients) during a medical procedure or a part thereof. Invasive patient interfaces (such as an endotracheal tube (ETT), tracheostomy tube, laryngeal mask airway (LMA) etc.) are used to penetrate the patient's airway and provide ventilatory support e.g., by providing cyclic inspiratory and expiratory phases of lung inflation and deflation for oxygenation and pressure support when the patient is apnoeic.

Invasive respiratory devices such as ETTs and tracheostomy tubes could also be used to provide respiratory support in the absence of cycles of lung inflation and deflation corresponding to mechanical ventilation. The safe duration of respiratory support can be limited by how quickly the patient's blood O₂ becomes depleted and/or how quickly the patient's blood CO₂ builds up, which are affected by the level of oxygenation and/or carbon dioxide clearance being achieved in the lungs. Non-mechanical ventilation methods and systems could exacerbate these issues and/or be faced with other issues such as increased barotrauma risk.

It may be desirable to improve provision of respiratory support that addresses some shortcomings of existing solutions of providing ventilatory support to apnoeic patients.

A reference herein to a patent document or any other matter identified as prior art, is not to be taken as an admission that the document or other matter was known or that the information it contains was part of the common general knowledge as at the priority date of any of the claims.

### SUMMARY OF INVENTION

Viewed from one aspect, the present disclosure provides a connector for coupling with an invasive patient interface providing respiratory support to a patient, the connector comprising: (a) a connector body having (i) an inlet port couplable with a flow source providing a flow of respiratory gas; (ii) a gases exit port; and (iii) a device port couplable with the invasive patient interface; and (b) a lumen having a first end and a second end; wherein the connector body defines a gas flow path between the inlet port and both the device port and the lumen first end; and wherein the lumen second end is disposed outside the device port.

In some embodiments, the lumen second end is disposed outside the device port for delivery of gas at a target location deeper in the patient's airway than the device port.

The connector body may be couplable, directly or indirectly via an adapter, with an invasive patient interface. Coupling may include feeding the lumen into the invasive patient interface before coupling the device port with a corresponding port of the invasive patient interface. In some embodiments, the coupling may be releasable to provide for repeated coupling and decoupling of the connector and the invasive patient interface, e.g. to connect with different flow sources and/or systems to provide different forms of respiratory support.

In some embodiments, the lumen provides an outflow path for gases entering the lumen second end at a target location deeper in the patient's airway than the device port. In some embodiments, the lumen first end may be configured to provide for gases in the outflow path to flow to surrounding atmosphere.

In some embodiments, the lumen second end is disposed, when in use, between the device port and a proximal end of the invasive patient interface.

The target location may be within the invasive patient interface, close to the proximal end of the invasive patient interface although that need not be the case and in some embodiments, the target location may be provided outside the invasive patient interface by the lumen extending beyond the proximal opening of the invasive patient interface through which gases may be delivered.

In some embodiments, the entire lumen is integral with the connector body. In other embodiments, at least part of the lumen is integral with the connector body and the lumen comprises an elongate tube which, when in use, extends through at least part of the connector body. The elongate tube may be trimmed to a predetermined or required length. In some embodiments, the elongate tube may include one or more markings discernible from outside the connector body, designating proximity of the lumen second end to the tip of the invasive patient interface.

In some embodiments, the connector body may comprise a guide portion for positioning the elongate tube relative to the connector body. The guide portion may comprise a feature selected from a group including but not limited to e.g. (a) a channel; (b) an eyelet; and (c) a groove or slot. In some embodiments, the guide portion may be disposed on an internal wall of the connector body defining a flow path from the inlet port to the device port. Alternatively/additionally, the guide portion may be disposed on an internal wall of the connector body defining a flow path from the device port to the gases exit port. Alternatively, the guide portion (or part thereof) may be disposed externally of the connector body. In some embodiments, the guide portion may be integral with the connector body.

In some embodiments, the connector body may comprise a locating feature for preventing the lumen from protruding out of the invasive patient interface when in use. In some embodiments, the locating feature may comprise an engagement structure adapted to couple with a cooperating structure on the invasive patient interface or an adapter connected to the invasive patient interface. The engagement structure and the cooperating structure may be respectively located to maintain a set back of the lumen second end within the invasive patient interface.

In some embodiments, the connector body defines a gas outflow path between the device port and the gases exit port. In some embodiments, the connector body defines a gas outflow path between the inlet port, the device port and the gases exit port.

In some embodiments, the connector is configured to generate a predetermined airway pressure when coupled with the invasive patient interface in use. The predetermined pressure may be at least about 2 cm H₂O. In some embodiments, the predetermined pressure may be in a range of about 2 cm H₂O about 20 cm H₂O, preferably about 2 cm H₂O to about 10 cm H₂O, and more preferably about 2 cm H₂O to about 5 cm H₂O. In some embodiments, the flow of respiratory gas into the inlet port is divided between at least the device port and the lumen achieving a flow state in which airway pressure is maintained at the predetermined pressure.

In some embodiments, the connector includes one or more constrictions configured to generate the predetermined airway pressure. Preferably, the one or more constrictions are disposed between the inlet port and the device port. In some embodiments, the one or more constrictions are disposed upstream of where a gas flow path between the inlet port and the device port meets a gas outflow flow path between the device port and the gases exit port.

In some embodiments, the flow of respiratory gas into the inlet port is divided between at least the device port and the lumen, achieving a flow state in which respiratory gas is delivered to the target location at, at least, a first predetermined velocity sufficient to achieve enhanced CO₂ clearance.

In some embodiments, the flow of respiratory gas into the inlet port may be divided between the device port, the lumen and the gases exit port.

In some embodiments, the flow of respiratory gas into the inlet port is divided only between the device port and the gases exit port achieving a flow state in which airway pressure is maintained at the predetermined pressure. Preferably the flow state achieves removal of gases from the target location at a second predetermined velocity of flow within the lumen sufficient to achieve enhanced CO₂ clearance.

In some embodiments, the connector has a lumen with dimensions configured to achieve one or both of the first and second predetermined velocity at the target location. In some embodiments, the lumen has an internal diameter which, relative to the internal diameter of the invasive patient interface, achieves one or both of the first and second predetermined velocity during use. In some embodiments, the lumen has an internal cross-sectional area which, relative to the internal cross-sectional area of the invasive patient interface, achieves one or both of the first and second predetermined velocity during use. In some embodiments, one or both of the first and second predetermined velocity may be about 5 m/s to about 25 m/s, preferably about 5 m/s to about 15 m/s.

In some embodiments, the lumen has an internal diameter of about 2 mm to about 5 mm.

In some embodiments, the inlet port receives a flow of respiratory gas having a flow rate in a range of about 10 LPM to about 150 LPM, preferably about 20 LPM to about 70 LPM.

In some embodiments, a filter may be configured to treat gases in the gas outflow path.

Preferably, the invasive patient interface is a sealing interface selected from a group including but not limited to (a) an endotracheal tube; (b) a laryngeal mask airway; (c) a tracheostomy tube; and (d) a suspension laryngoscope.

Viewed from another aspect, the present disclosure provides a system for providing respiratory support to a patient, the system comprising (a) a flow source providing a flow of respiratory gas, (b) the connector according to any one of the embodiments disclosed in the context of the previous aspect; and (c) an invasive patient interface.

In some embodiments, the connector and the invasive patient interface may be integrally formed or provided as a single part.

In some embodiments, the flow source provides flows of respiratory gas at a flow rate in a range of about 0 LPM to about 150 LPM, preferably about 20 LPM to about 70 LPM.

Viewed from another aspect, the present disclosure provides a system for providing respiratory support to a patient using an invasive patient interface, the system comprising: (a) a flow source providing a flow of respiratory gas; (b) a connector having an inlet port receiving a flow of respiratory gas from the flow source, a device port for coupling with the invasive patient interface, and gases exit port; and (c) a lumen having a second end configured to be disposed at a target location inside the invasive patient interface; wherein the connector defines a first gas flow path for at least a portion of the flow of respiratory gas entering the lumen to the target location and an exit flow path for escape of gases exiting the airway through the device port; and wherein the system provides a flow of respiratory gas through the lumen second end at a first predetermined velocity thereby increasing removal of CO₂ through the invasive patient interface for exit via the gases exit port.

In some embodiments, the target location is within the invasive patient interface, preferably close to the proximal end of the invasive patient interface. However, that that need not always be the case and in some embodiments, the target location may be outside the invasive patient interface.

In some embodiments, the flow of gas provided by the flow source is substantially continuous. In some embodiments, the flow rate of gas provided by the flow source is in a range of 0 to about 150 LPM, preferably about 20 LPM to about 70 LPM. In some embodiments, the flow rate of gas provided by the flow source is at least about 40 LPM.

In some embodiments, all flow from the flow source is directed to the first gas flow path and delivered to the target location.

In some embodiments, the connector provides a gas flow path for delivery of a portion of the flow of respiratory gas to the invasive patient interface.

It is to be understood that several factors contribute to enhanced CO₂ clearance in various embodiments and aspects of the disclosure and these factors can interrelate. One factor is the velocity of gases at a target location within the patient's airway. Increasing the velocity of gases at a target location within the patient's airway increases CO₂ clearance. Within a patient's airway includes within the invasive patient interface, at an end of the invasive patient interface, and outside of the invasive patient interface but within the patient's airway (for example between the end of the invasive patient interface and the patient's carina). In some configurations, the target location is at or close to the carina. The velocity of gases at the target location may be based on one or more exit velocities (e.g. average velocity) of gases exiting the lumen second end for delivery to the target location, and the distance of the lumen second end from the target location (e.g. the carina). The exit velocity of gases exiting the lumen second end may be influenced by factors such as flow rates at which gases are provided to the inlet port, the resistance to flow of the flow path in which gases flow to the patient (e.g. cross sectional area of the flow path and/or the geometry of the internal walls of the lumen defining the flow path and/or the length of the flow path, and/or the presence or absence of constrictions in the flow path), the pressure gradient across which the gases flow, the flow rate of gases exiting the gases exit port, the velocity of gases exiting the gases exit port and the resistance to flow through the gases exit port. In some embodiments, the velocity of gases exiting the gases exit port may be influenced by the exit velocity of gases exiting the lumen second end. In some scenarios, a desirable exit velocity of gases exiting the lumen second end is achieved about 5 mm from the carina since there is a risk of damage if gases are delivered too close to or at the carina. In most medical procedures however, a clinician locates the proximal end of the invasive patient interface (e.g. ETT) about 4 cm from the carina which reduces the likelihood of gases being delivered too close to or at the carina. Suitable exit velocities may include e.g. a velocity in a range of above about 0 m/s to about 25 m/s at a selected flow rate of about 5 L/min to about 70 L/min of the flow of gas provided to the lumen, such as a velocity in the range of about 5 m/s to about 20 m/s. In some embodiments, the exit velocity is a velocity in a range of about 10 m/s to about 15 m/s.

Another factor that contributes to enhanced CO₂ clearance in various embodiments and aspects of the disclosure is the flow rate of gases exiting the patient. In some configurations, this flow rate may be the flow rate of gases exiting via the gases exit port. Increasing this flow rate of gases exiting the patient increases CO₂ clearance. The flow rate at which gases exit the patient is related to the flow rate at which gases are provided to the patient, for example through the lumen second end. The resistance to flow within the connector and/or delivery lumen (e.g. lumen 260) may affect the flow rate delivered to the patient. In some embodiments, for a given driving pressure of the flow source, altering the resistance to flow in the connector and/or delivery lumen may alter the flow rate being delivered to the patient and hence alter the flow rate exiting the patient via the gas outflow path. Resistance to flow can be influenced by parameters of the flow path in which gases flow to the patient (e.g. cross sectional area of the flow path and/or the geometry of the internal walls of the lumen defining the flow path and/or the length of the flow path). As indicated above, a flow rate of gases provided by the flow source may be in a range of 0 to about 150 LPM, preferably about 20 LPM to about 70 LPM. In some embodiments, the flow rate of gas provided by the flow source is at least about 40 LPM and in many applications is about 70 LPM. Part of or all of the flow rate of gases provided by the flow source may be provided to the patient (via the lumen). In some configurations, the flow rate of gases exiting the patient is inversely related to the resistance to flow of the exit flow path from the patient to atmosphere, for example, the higher the resistance to flow, the lower the flow rate. This resistance to flow can be influenced by parameters of the exit flow path from the patient to atmosphere (e.g. cross sectional area of the flow path and/or the geometry of the internal walls of the lumen defining the flow path and/or the length of the flow path). The resistance to flow of the exit flow path from the patient to atmosphere is related to pressures delivered to the patient, for example the greater the resistance to flow in the exit flow path, the greater the delivered pressures.

Another factor that contributes to enhanced CO₂ clearance in various embodiments and aspects of the disclosure is the pressure differential between inside the patient's airway and atmosphere. A pressure differential can affect flow rate of gases exiting the patient's airway, where a greater pressure gradient will give rise to a greater flow rate of gases exiting the patient's airway. The pressure differential between inside the patient's airway and atmosphere may be influenced by factors such as flow rates at which gases are provided to the inlet port, the resistance to flow of the flow path in which gases flow to the patient (e.g. cross sectional area of the flow path and/or the geometry of the internal walls of the lumen defining the flow path and/or the length of the flow path, and/or the presence or absence of constrictions in the flow path), the flow rate of gases exiting the gases exit port, the velocity of gases exiting the gases exit port and the resistance to flow through the gases exit port. The pressure differential may be determined by measuring the difference between pressure at the lumen second end and pressure within the connector body, or the difference between pressure at the lumen second end and atmosphere (as may be measured at or outside the gases exit port). Pressure measurements may be obtained using pressure sensors at these locations.

In some embodiments, the entire lumen is integral with the connector body. In other embodiments, at least part of the lumen is integral with the connector body.

In some embodiments, the system comprises an invasive patient interface. The connector and the invasive patient interface may be provided as separate components, or they may be integrally formed or provided as a single part.

In some embodiments, the connector comprises a locating feature for preventing the lumen from protruding out of the invasive patient interface when in use. In some embodiments, the locating feature may comprise an engagement structure adapted to releasably couple with a cooperating structure on the invasive patient interface or an adapter connected to the invasive patient interface, the engagement structure and cooperating structure being respectively located to maintain a set back of the lumen second end within the invasive patient interface.

In some embodiments, the system comprises a filter configured to treat gases in the exit flow path.

In some embodiments, the respiratory support provided by the system maintains a period of safe apnoea. Alternatively/additionally, the respiratory support provided by the system causes little or no movement of the patient.

In some embodiments, the system includes a controller controlling the flow source. In some embodiments, the controller may receive inputs indicative of velocity at the lumen second end, or at the target location, or inputs indicative of flow rate in the exit flow path and adjust control of the flow source to achieve the first predetermined velocity and/or flow rate through the lumen second end. Alternatively/additionally, the controller may receive inputs indicative of a flow rate of gases delivered to the connector (e.g. at the inlet port) or in the exit flow path and adjust control of the flow source to achieve removal of gases from the target location at a second predetermined velocity or at a flow rate in the exit flow path that increases CO₂ clearance. Alternatively/additionally, the controller may receive inputs indicative of a pressure gradient between the lumen second end (or the target location) and atmosphere, and adjust control of the flow source and/or a pressure source (such as a negative pressure source) to achieve removal of gases from the target location that increases CO₂ clearance. Alternatively/additionally the controller may receive inputs indicative of pressure in the patient's airway and adjust control of the flow source to achieve a predetermined patient airway pressure. The predetermined airway pressure may be at least about 2 cmH₂O for example in a range of about 2 cmH₂O to about 20 cmH₂O, preferably about 2 cmH₂O to about 10 cmH₂O, and more preferably about 2 cmH₂O to about 5 cmH₂O.

In some embodiments, the controller may be configurable to receive inputs comprising signals generated by one or more of: (a) an expired gas flow sensor; (b) an airway pressure sensor; (c) a movement sensor; and (d) a user input device.

In some embodiments, inputs received by the controller may include a breathing input indicative of spontaneous breathing by the patient and wherein the controller controls the flow source to deliver respiratory support only when the breathing input indicates an absence of spontaneous breathing by the patient. Alternatively/additionally, inputs received by the controller may include a movement input indicative of movement of the patient during delivery of the respiratory support, and wherein the controller generates a control signal to produce an audible and/or visible and/or haptic output indicating substantial absence of movement during delivery of respiratory support by the system.

In some embodiments, the system comprises a movement sensor configured to detect movement, such as breathing movement or movement of the thorax and/or abdomen of the patient during delivery of the respiratory support.

In some embodiments, the system may comprise one or more of a display device, a speaker output module and a haptic feedback module operable by the controller to provide an output discernible by a clinician while performing a medical procedure on the patient.

In some embodiments, the system comprises the invasive patient interface which is preferably a sealing interface. The invasive patient interface may be selected from a group including but not limited to e.g.: an endotracheal tube, a laryngeal mask airway, a tracheostomy tube and a suspension laryngoscope. In some embodiments, the connector and the invasive patient interface may be integrally formed, or provided as a single part.

In some embodiments, the system comprises a humidifier configured to condition the gas to a pre-determined temperature and/or humidity before delivery to the patient.

In some embodiments, the system comprises a filter configured to treat expired gas from the patient before it is released to atmosphere.

In some embodiments, the system comprises a gas delivery conduit providing a flow of gas from the flow source to the invasive patient interface.

In some embodiments, the respiratory support is delivered by the system during absence of spontaneous breathing.

In some embodiments, the respiratory support is deliverable by the system during a medical procedure requiring substantial stillness of the abdomen and/or thorax.

Viewed from another aspect, the present disclosure provides a method for providing respiratory support to a patient, comprising providing a flow of respiratory gas from a single gas source to a connector inlet that is in fluid communication with a device port and a lumen, wherein the device port is coupled with an invasive patient interface delivering a portion of the respiratory gas to the patient's airway, and wherein the lumen has a second opening delivering a portion of the respiratory gas to a target location within the invasive patient interface that is located proximally of the device port.

Viewed from another aspect, the present disclosure provides a method for providing respiratory support to a patient via an invasive patient interface, comprising providing a flow of respiratory gas from a flow source to the connector disclosed in relation to any one of the embodiments of the foregoing aspects.

In some embodiments of the above aspects, the method causes little or no movement of the patient.

In some embodiments of the above aspects, the method comprises the step of, prior to providing the flow of respiratory gas to the connector, inducing the patient into a state of general anaesthesia.

Viewed from another aspect, the present disclosure provides a connector for coupling with an invasive patient interface providing respiratory support to a patient, the connector comprising: (a) a connector body having: (i) an inlet port couplable with a flow source providing a flow of respiratory gas; (ii) a gases exit port; (iii) a device port couplable with the invasive patient interface; and (iv) a gases sampling port; and (b) a lumen having a first end and a second end; wherein the connector body defines a gas flow path between the inlet port and both the device port and the lumen inlet; and wherein the lumen second end is disposed outside the device port.

In some embodiments, the gases sampling port is configured to facilitate fluid coupling of sampled gases from a sampling location to an instrument for analysing sampled gases, such as e.g. a capnography machine for analysing sampled CO₂ gas. In some embodiments, the gases sampling port is couplable with a conduit providing fluid communication of sampled gases from the gases sampling line to the instrument.

In some embodiments, the gases sampling port provides fluid communication with or receives a gases sampling line having a sampling end which is locatable within the invasive patient interface for sampling gases at a sampling location. In some embodiments, at least part of the gases sampling line may be integral with the connector body. Alternatively/additionally, at least part of the gases sampling line may be integral with the connector lumen.

In some embodiments, the gases sampling port is configured to receive insertion of a gases sampling line through the connector body, the gases sampling line having a first end which is couplable with the instrument, and a second end which is locatable within the invasive patient interface for sampling gases at a sampling location which in some embodiments, may be within the patient's airway.

In some embodiments, the sampling location is inside the invasive patient interface when within the patient's airway. The sampling location may be longitudinally offset with respect to the lumen second end for example it may be offset distally of the lumen second end (i.e. set back inside the invasive patient interface, relative to the lumen second end). Alternatively/additionally, the sampling location may be inside the connector body.

Viewed from another aspect, the present disclosure provides a connector for coupling with an invasive patient interface providing respiratory support to a patient, the connector comprising: (a) a connector body having: (i) an inlet port couplable with a flow source providing a flow of respiratory gas; (ii) a gases exit port; and (iii) a device port couplable with the invasive patient interface; (b) a lumen having a first end and a second end; and (c) a variable aperture for adjusting flow of gases exiting the connector through the gases exit port; wherein the connector body defines a gas flow path between the inlet port and both the device port and the lumen inlet; and wherein the lumen second end is disposed outside the device port.

In some embodiments, the connector includes a cap applied to or formed over an opening in the gases exit port, the cap having a first member with a first opening and a second member with a second opening, wherein relative movement between the first member and the second member varies an amount of overlap between the first and second openings to define the variable aperture. In some embodiments, one of the first member and the second member is stationery in use, and the other of the first member and the second member is movable relative to the stationery member. Relative movement between the first member and the second member may be rotational although that need not be the case and translational or other relative movements may be provided.

In some embodiments, the connector body has a first opening in a wall portion defining the gases exit port, and the connector further comprises a movable collar arranged around at least part of the wall portion defining the exit gases port, the collar having a second opening, wherein movement of the collar varies an amount of overlap between the first and second openings to define the variable aperture.

In some embodiments, the collar may be rotatable around the wall portion defining the exit gases port. In other embodiments, the collar may be translationally moveable along the wall portion defining the exit gases port.

In some embodiments, the connector comprises a connector body extension providing the variable aperture.

Viewed from another aspect, the present disclosure provides a connector assembly for coupling with an invasive patient interface providing respiratory support to a patient, the connector assembly comprising: (a) a connector having (i) an inlet port couplable with a flow source providing a flow of respiratory gas; (ii) a gases exit port; and (iii) a device port couplable with the invasive patient interface; and (b) a lumen defined by a catheter, the lumen having a first end and a second end; wherein the connector defines a gas flow path between the inlet port and both the device port and the lumen first end; and wherein the lumen second end is disposed outside the device port.

It is to be understood that various features and aspects of the disclosed embodiments can be combined with or substituted for one another in order to form varying modes of the disclosed embodiments. Thus, it is intended that the scope of the present disclosure should not be limited by the particular aspects and feature combinations expressly disclosed, but should be determined as encompassing feature combinations not expressly disclosed but nevertheless understood upon fair reading of the specification to be suitable for combination in a manner similar to other aspects and embodiments disclosed elsewhere herein.

### BRIEF DESCRIPTION OF DRAWINGS

The invention will now be described in greater detail with reference to the accompanying drawings in which like features are represented by like numerals. It is to be understood that the embodiments shown are examples only and are not to be taken as limiting the scope of the invention as defined in the claims appended hereto.

FIG. 1 is a schematic illustration of a system for providing respiratory support to a patient utilising connectors according to various embodiments of the disclosure.

FIG. 2 is a cross sectional view of a connector for coupling with an invasive patient interface according to an embodiment of the disclosure.

FIG. 3 is a cross sectional view of a modified version of the connector in FIG. 2.

FIG. 4 is an end view of a connector showing constrictions according to an embodiment of the disclosure.

FIG. 5 is a cross sectional view of an alternative embodiment of a connector providing an exit lumen according to an embodiment of the disclosure.

FIG. 6 is a cross sectional view of a connector according to another embodiment of the disclosure

FIG. 7 is a cross sectional view of a modified version of the connector in FIG. 6.

FIGS. 8A and 8B are schematic illustrations of a connector with a locating feature in the form of a protrusion formed on an internal wall of connector body.

FIGS. 9A and 9B are sectional and perspective views respectively of an alternative connector in which the gases exit port is provided by multiple openings.

FIGS. 10A, 10B and 10C are schematic illustrations showing a variable aperture which may be incorporated into a connector according to an embodiment of the disclosure.

FIGS. 11A, 11B, 11C and 11D are schematic illustrations showing a variable aperture which may be incorporated into a connector according to another embodiment of the disclosure.

FIG. 12 is a cross sectional view of a connector for coupling with an invasive patient interface, providing a gases sampling port, according to an embodiment of the disclosure.

### DETAILED DESCRIPTION

Embodiments of the disclosure are discussed herein by reference to the drawings which are not to scale and are intended merely to assist with explanation of the invention.

Embodiments of the disclosure are generally directed to provision of respiratory support to a patient via an invasive patient interface, such as an endotracheal tube (ETT), laryngeal mask airway (LMA), a tracheostomy tube or suspension laryngoscope. Embodiments of the disclosure may be used during medical procedures in which the patient is sedated and for which respiratory stillness is required. The respiratory support provides a flow of gases from a flow source to the patient's airway, via the invasive patient interface. For the embodiments disclosed, the respiratory support is provided at flow rates and velocities to clear CO₂ from the patient and/or to provide pressure support to the patient and/or to oxygenate the patient. A lumen of smaller cross sectional area relative to the cross-sectional area of the invasive patient interface is also provided, with a proximal end at a target location, for achieving improved CO₂ clearance and/or oxygenation and/or pressure support.

Embodiments of the disclosure aim to deliver respiratory support by employing a system that includes a connector configured to produce one or more jet flows of gas, optionally in and/or through the connector. The one or more jet flows of gas may flow to an invasive patient interface (such as an ETT) coupled with the connector. The system delivers inspiratory flow from a flow source, optionally via a humidifier, to the connector. The connector is configured to receive the inspiratory flow, and to deliver one or more jet flows of gas through one or more outlets of the connector. One or more of the jet flows of gas through the connector may flow towards the invasive patient interface and patient.

A first jet flow of gas may be provided by the connector as a result of parameters of the connector including the total cross-sectional area of one or more constrictions in the connector and/or other parameters such as the flow rate of gas received at the connector or the distance from the one or more constrictions to the outflow opening (device port) through which the gas flows into the invasive patient interface. In some embodiments, the connector includes one or more flow constrictions for providing the first jet flow of gas. The one or more flow constrictions are preferably disposed in the inspiratory flow path to provide the jet flow of gas towards the invasive patient interface while also providing an outflow path for the jet flow of gas via the gases exit port. The one or more flow constrictions may include, for example, a nozzle, a tapered region for constricting the flow of gas, and/or a plurality of apertures or openings through which the first jet flow of gas is delivered. A gas outflow path permits gas flow from the patient to atmosphere, and from the one or more constrictions to atmosphere, via the gases exit port. A filter may be provided to treat gases in the gas outflow path before they are released to atmosphere.

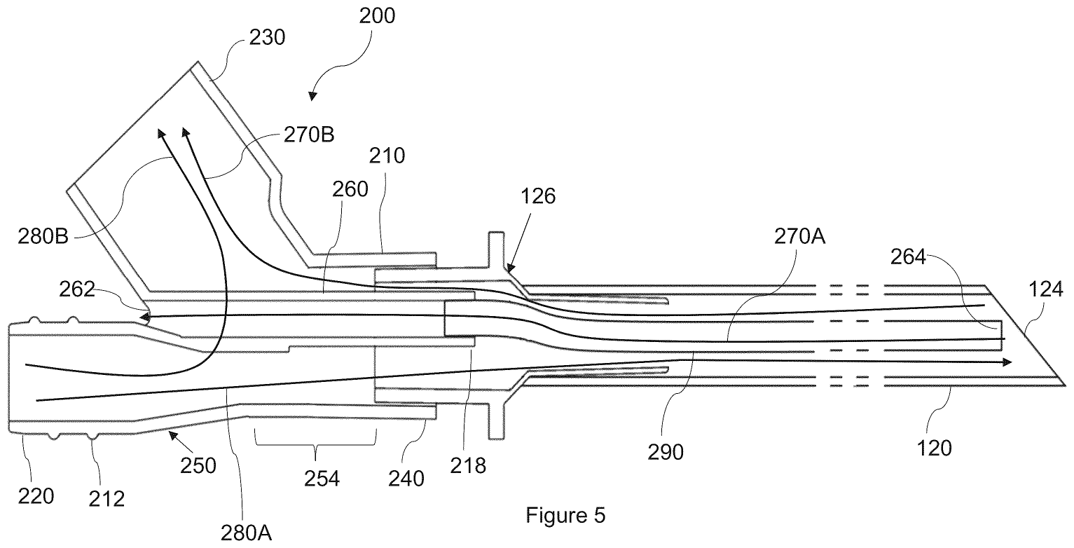

The size, number and shape of the one or more constrictions can be optimised to provide desirable characteristics of the first jet flow of the gas for providing respiratory support for a given flow rate into the connector. The one or more constrictions may be in a tapered or untapered nozzle having a plurality of openings or apertures as the outlet. In these embodiments, the increase in velocity to form the first jet may be due to the decreased total cross-sectional area of the openings comprising the flow constrictions, relative to the larger diameters of the inlet port into which the respiratory gas is provided from the flow source. Thus, a person skilled in the art would readily appreciate that embodiments of the disclosure are not limited to requiring nozzles or tapered regions in order to provide the one or more flow constrictions. In some embodiments, the one or more flow constrictions comprise a nozzle, as described in relation to FIG. 5.

In some embodiments, the connector includes a lumen for providing a second jet flow of gas. The lumen has an outlet end through which the second jet flow of gas is delivered to a target location in a patient.

A jet flow of gas is a region of high velocity of gas, or a gas with a velocity higher than the average velocity of gas flow in the connector and/or elsewhere in the system (for example a gas adjacent or proximal to the jet flow). In some configurations, the jet flow of gas includes a region of high velocity of gas exiting an end of a lumen. The one or more jet flows of gas are preferably at least at a predetermined velocity that is capable of achieving one or more objectives such as increased pressure support and/or oxygenation to the patient by increasing patient pressure, i.e. pressure in the airway, enhanced gas mixing in the patient's airway and/or CO₂ clearance from the patient airway, optionally at a target location. It is to be understood that the term “velocity” used in the specification herein includes an average velocity which is an average of the velocities of a gas flow across a cross-sectional area of a flow path.

The first jet flow of gas preferably includes a velocity that is capable of achieving or contributing a target patient pressure of at least 2 cmH₂O near the outlet of the invasive patient interface, or more preferably utilising a connector according to certain embodiments disclosed herein, near the lumen outlet (second) end, or in the patient's airway when in use. As noted above, other factors that can contribute to target patient pressures include the resistance to flow of the flow path in which gases exit the patient. This resistance to flow can be influenced by parameters of the exit flow path from the patient to atmosphere (e.g. cross sectional area of the flow path and/or the geometry of the internal walls of the lumen defining the flow path and/or the length of the flow path). The resistance to flow of the exit flow path from the patient to atmosphere is related to pressures delivered to the patient, for example the greater the resistance to flow in the exit flow path, the greater the delivered pressures.

The velocity of the first jet flow of gas may be greater or less than the velocity of a gases flow provided or generated by a flow source 110. Preferably, the velocity of the first jet flow of gas is greater than the velocity of a gases flow provided or generated by a flow source 110. Preferably, the first jet flow of gas includes a velocity in a range of about 5 m/s to about 60 m/s. Preferably, the first jet flow of gas includes a velocity in a range of about 5 m/s to about 60 m/s at a selected flow rate of about 20 L/min to about 70 L/min of the flow of gas provided by the flow source 110.

A second jet flow of gas provided to a target location preferably includes a velocity that is capable of achieving enhanced gas mixing and improved CO₂ clearance at the target location when in use. In some embodiments, the velocity of the second jet flow of gas is greater than the velocity of a gases flow provided or generated by a flow source 110. Preferably, the second jet flow of gas includes a velocity in a range of above about 0 m/s to about 25 m/s. Preferably, the second jet flow of gas includes a velocity in the range of about 5 m/s to about 20 m/s. In some embodiments, the second jet flow of gas includes a velocity in a range of about 10 m/s to about 15 m/s at a selected flow rate of about 5 L/min to about 70 L/min of the flow of gas provided by the flow source 110. In embodiments having a lumen that provides the only gas flow path delivering all of the flow of respiratory gas to the target location, a second jet flow of gas is provided at the lumen second end. There is no first jet flow of gas provided by constrictions in the connector.

Embodiments of the disclosure are directed to a system configured to provide respiratory support to a subject by generating a pressure within a range of desirable patient pressures for a given range of flow rates. Desirable patient pressures for providing respiratory support may include a pressure or a range of pressures that are capable of achieving and/or maintaining a patent patient airway, assisting with lung recruitment, preventing or mitigating atelectasis and/or reducing the work of breathing.

Without being limited by theory, use of the lumen or catheter as disclosed herein to either deliver a portion of the respiratory support including the second jet flow of gas to a target location at a predetermined velocity, or to remove gases from the target location at a predetermined velocity and/or flow rate, enhances gas mixing at the target location which in turn improves CO₂ clearance from the patient. Connectors according to embodiments of the present disclosure utilise a lumen having a proximal end at the target location deep in the patient's airway to reduce the dead space of the flow path so that the gas mixing effect can be achieved deep in the patient's airway, ideally at a target location that is close to the patient's carina or deeper in the patient's bifurcated airway. This reduction in the dead space of the flow path due to the lumen and the location of the proximal end of the lumen can contribute to improved CO₂ clearance and in turn, a reduction in the rate of CO₂ build up in the patient's airway and blood. In addition, the reduction in the dead space of the flow path due to the lumen and the location of the proximal end of the lumen can contribute to improved delivery of oxygenated gas to the patient's lungs and in turn, improved O₂ levels in the patient's airway and blood. Improved O₂ levels in the patient's airway and/or blood can also be achieved with increased pressure delivery which may help to prevent, reduce or reverse atelectasis.

In some embodiments, gas mixing is caused by movement of the gas exiting the lumen at the predetermined velocity (or faster) which creates gas separation at the proximal end of the lumen. This generates turbulence that induces gas mixing at the target location which results in fresh gas delivered from the connector mixing with old and expired gas (being gas that has been expired from the lungs) in the patient's airway. Increasing the velocity of a flow of gases can generate more turbulence and more gas mixing at the target location. The connector also provides a gases outflow path for removal of the mixed gases at a rate that is determined in part by the dimensions of the flow paths defined by the connector body, and the rate of flow at which respiratory gas enters the inlet port.

Owing to the characteristics of the respiratory support being delivered via the invasive patient interface, a patient pressure is generated within the patient's airways which exceeds atmospheric pressure. In embodiments which may be described as providing an “exit lumen”, the proximal end of the lumen defines an entry opening for gases near the target location to flow down a pressure gradient to atmosphere via a gas outflow path defined by the lumen. This provides a mechanism for removal of old and expired gas (being gas that has been expired from the lungs) in the patient's airway and contributes to clearance of CO₂ rich gas. CO₂ clearance may be improved by increasing the pressure gradient. In some embodiments, a pressure gradient of from about 20 cmH₂O to above 0 cmH₂O, preferably about 10 cmH₂O to above 0 cmH₂O is desired and in some embodiments, a pressure gradient of about 5 cmH₂O may be preferred. The pressure gradient may be determined with reference to atmosphere in embodiments where the lumen provides a gas outflow path to atmosphere, or to another reference value determined by e.g. a pressure source, typically a negative pressure source, which may be in fluid communication with the lumen first end. CO₂ clearance may also be increased by increasing the cross-sectional area of the gas outflow path defined by the lumen.

An absence of movement of the diaphragm and/or thorax and/or abdomen may be termed ‘respiratory stillness’. However, the safe duration of a respiratory support during a period of respiratory stillness may be limited by CO₂ build up in the patient's blood which can be affected by CO₂ clearance in the lungs. In spontaneously breathing patients, the movement of the diaphragm inflates and delates the lungs, where the deflation of the lungs creates expiratory movement of the gases in the patient's airway to clear CO₂. Mechanical ventilation can support ventilation in spontaneously breathing patients, for example a respiratory support system could deliver a set pressure or volume of gases based on the patient's breathing pattern. In non-spontaneously breathing patients supported by mechanical ventilation, breathing is mimicked and the control of pressure and/or volume of the mimicked expiratory phase allows for the clearance of CO₂ from the patient's airways, predominantly caused by elastic recoil from the previously inflated lungs. In the absence of an expiratory motion, whether via spontaneous breathing (with or without mechanical ventilator support) or via mechanical ventilation in a non-spontaneously breathing patient, CO₂ could build up in the patient because of inadequate gas mixing leading to insufficient CO₂ clearance.

Beneficially, CO₂ clearance is achieved while the patient is substantially still or the at least the patient's thoracic region is still, and delivery of respiratory support according to the embodiments disclosed herein causes little or no movement of the patient. Delivery of respiratory support during a period of what may be termed “respiratory stillness”, i.e. while the patient is apnoeic/in the absence of mechanical ventilation, may be advantageous during medical procedures requiring stillness of the abdomen and/or thorax although utility of the disclosure is not limited to these circumstances. While embodiments of the disclosure may be beneficial in prolonging periods of safe respiratory stillness, it is still possible to use the connectors disclosed herein to provide ventilatory respiratory support in which there is displacement of the diaphragm.

Advantageously, embodiments of the present disclosure permit provision of respiratory support in the absence of cycles of lung inflation and deflation (i.e. in the absence of ventilation involving significant movement of the diaphragm). Advantageously, embodiments of the present disclosure permit provision of respiratory support during a period of respiratory stillness. This provision of respiratory support occurs by providing oxygenation, pressure support and/or CO₂ clearance. The CO₂ clearance benefits of embodiments disclosed herein are especially important for gas exchange during respiratory stillness as there is no breathing motion (inhalation/exhalation) to clear CO₂.

In this specification, invasive patient interfaces include any device or instrument that is couplable with an airway of the patient, usually bypassing the patient's upper respiratory tract and/or lower respiratory airway. Invasive patient interfaces may include but are not limited to devices and instruments that penetrate via a patient's mouth, nose or skin to serve as an artificial airway, such as an endotracheal tube, tracheostomy tube, laryngeal mask, suspension laryngoscope, or endoscope, to name a few. It will be appreciated that these are examples only, and that embodiments of the disclosure are not limited to use with endotracheal tubes or particular invasive patient interfaces described herein, and may employ other patient interfaces as would be understood by a person skilled in the art.

In this specification, the terms subject and patient may be used interchangeably. A subject or patient may refer to a human or an animal subject or patient.

In this specification, the terms “distal” and “proximal” are to be interpreted relative to the patient. Distal refers to a feature being directed away from or further from the patient. Proximal refers to a feature being directed towards or close to the patient.

In this specification, the gas delivered by a flow source could include, without limitation, oxygen, carbon dioxide, nitrogen, helium, and anaesthetic agents, to name a few, or mixtures of these or other breathable gases for respiration and/or ventilation. Where reference is made to a particular gas herein, it will be appreciated that it is by way of example only and the description can apply to any gas—not just that referenced.

It is to be understood that the flow of respiratory gases provided to the patient may be humidified or non-humidified.

Without limitation, some indicative values of flow rates for the respiratory gas provided by a flow source can be as follows.

In some configurations, the respiratory support includes delivery of gases from a flow source to a connector at a flow rate of greater than 0 litres per minute (greater than 0 LPM or L/min). In some configurations, the respiratory support includes delivery of gases from a flow source to a connector at a flow rate of about 5 or 10 LPM to about 150 LPM, or about 10 LPM to about 120 LPM, or about 15 LPM to about 95 LPM, or about 20 LPM to about 90 LPM, or about 20 LPM to about 70 LPM, or about 25 LPM to about 85 LPM, or about 30 LPM to about 80 LPM, or about 35 LPM to about 75 LPM, or about 40 LPM to about 70 LPM, or about 45 LPM to about 65 LPM, or about 50 LPM to about 60 LPM. For example, according to various embodiments and configurations described herein, a flow rate of gases supplied or provided to a connector of embodiments of the disclosure via a system or from a flow source, may comprise, but is not limited to, flows of at least about 5, 10, 15, 20, 30, 40, 50, 60, 70, 80, 90, 100, 110, 120, 130, 140, 150 LPM, or more, and useful ranges may be selected to be any of these values (for example, about 20 LPM to about 90 LPM, about 15 LPM to about 70 LPM, about 20 LPM to about 70 LPM, about 40 LPM to about 70 LPM, about 40 LPM to about 80 LPM, about 50 LPM to about 80 LPM, about 60 LPM to about 80 LPM, about 70 LPM to about 100 LPM, about 70 LPM to about 80 LPM).

In some configurations, the respiratory support includes delivery of gases to a patient at a flow rate of greater than 0 litres per minute (greater than 0 LPM or L/min). In some configurations, the respiratory support includes delivery of gases to a patient at a flow rate of about 5 or 10 LPM to about 150 LPM, or about 10 LPM to about 120 LPM, or about 15 LPM to about 95 LPM, or about 20 LPM to about 90 LPM, or about 20 LPM to about 70 LPM, or about 25 LPM to about 85 LPM, or about 30 LPM to about 80 LPM, or about 35 LPM to about 75 LPM, or about 40 LPM to about 70 LPM, or about 45 LPM to about 65 LPM, or about 50 LPM to about 60 LPM, or about above 0 LPM to about 10 LPM. For example, according to various embodiments and configurations described herein, a flow rate of gases supplied or provided via a connector of embodiments of the disclosure via a system or from a flow source, and to a patient, may comprise, but is not limited to, flows of at least about greater than 0 LPM, about 5, 10, 15, 20, 30, 40, 50, 60, 70, 80, 90, 100, 110, 120, 130, 140, 150 LPM, or more, and useful ranges may be selected to be any of these values (for example, about 20 LPM to about 90 LPM, about 15 LPM to about 70 LPM, about 20 LPM to about 70 LPM, about 40 LPM to about 70 LPM, about 40 LPM to about 80 LPM, about 50 LPM to about 80 LPM, about 60 LPM to about 80 LPM, about 70 LPM to about 100 LPM, about 70 LPM to about 80 LPM).

Flow rates at which gases are delivered from a flow source to a connector and/or delivered to a patient may be referred to as a therapeutic flow rate. The therapeutic flow rate can be time-varying (e.g. oscillating). That is, the therapeutic flow can have a time-varying (e.g. oscillating) flow rate component. This time-varying flow rate can help with therapy.

Flow rates for premature/infants/paediatrics (with body mass in the range of about 1 to about 30 kg) can be different. The flow rate can be set to about 0.4 L/min/kg to about 8 L/min/kg with a minimum of about 0.5 L/min and a maximum of about 70 L/min. For patients under 2 kg maximum flow rate is set to 8 L/min. Oscillating flow may be set to 0.05-2 L/min/kg with a preferred range of 0.1-1 L/min/kg and another preferred range of 0.2-0.8 L/min/kg.

In some embodiments, the flow rates disclosed herein may generate a flushing effect in the patient's airway such that the anatomical dead space of the upper airways is flushed by the incoming gas flows. This can create a reservoir of fresh gas, and/or minimise the gas concentration of carbon dioxide, nitrogen, etc. which may help to improve CO₂ clearance and/or reduce the build up of CO₂. Incoming gas flows with higher than ambient amount of oxygen may expedite the time required to replace gases, e.g. carbon dioxide, nitrogen, etc in the patient's lungs and/or increase the patient's blood oxygen saturation levels.

Gases delivered may comprise a percentage of oxygen. In some configurations, the percentage of oxygen in the gases delivered may be about 15% to about 100%, 20% to about 100%, or about 30% to about 100%, or about 40% to about 100%, or about 50% to about 100%, or about 60% to about 100%, or about 70% to about 100%, or about 80% to about 100%, or about 90% to about 100%, or about 100%, or 100%.

In some embodiments, a flow rate of gases supplied or provided to a connector of embodiments of the disclosure via a system or from a flow source, may generate a predetermined patient pressure of greater than 0 cmH₂O. The generated patient pressure may be between about 2 cmH₂O and about 20 cmH₂O, or about 2 cmH₂O to about 10 cmH₂O, or about 2 cmH_{2O} and about 5 cmH₂O, or about 5 cmH_{2O} to about 10 cmH₂O.

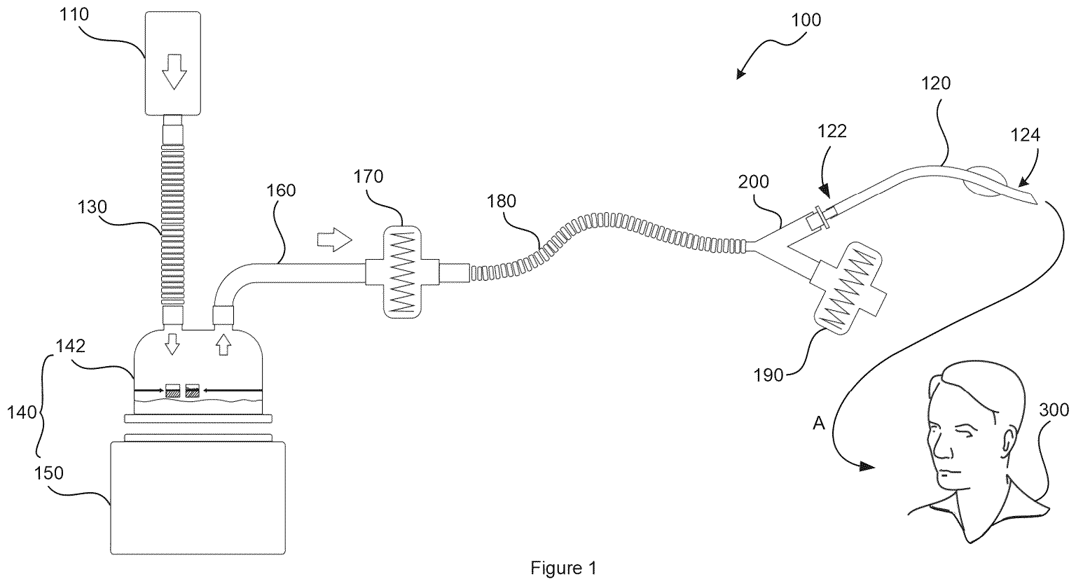

FIG. 1 is a schematic illustration showing a system 100 for providing respiratory support to a patient 300 according embodiments of the disclosure. System 100 includes a flow source 110 providing a flow of respiratory gas to a connector 200 and to the patient 300, in the direction of arrow A as indicated.

The gas flow is delivered from the flow source 110 to a conduit 130 connectable between the flow source 110 and a humidifier 140. The humidifier 140 includes a humidification chamber 142 and a humidification base unit 150. The conduit 130 may provide a dry line for delivering dry flow of gases to the humidifier 140. The conduit 130 may be coupled to the humidification chamber 142 of the humidifier 140 as shown. In alternative embodiments, the humidifier 140 may be a single component (not shown) and exclude the separate humidification chamber 142 and base unit 150. The humidifier 140 may be configured to condition the gas provided by the flow source 110 to a required temperature and/or humidity. The required temperature and/or humidity may be determined according to the respiratory support being delivered and may be selected by the user or operator to be suitable for the respiratory support to be provided.

The conditioned gas flow from the humidifier 140 (or more specifically the humidification chamber 142) travels through a conduit 160 to an inlet port 220 of the connector 200 for providing fluid communication to an invasive patient interface 120. In other embodiments, the system 100 may exclude any humidifier component and the flow source 110 may be directly couplable, or couplable through an interface conduit 180, to the inlet port 220 of the connector 200.

The flow source 110 may include a compressed gas source, a device that modifies flow from a compressed gas source and/or a flow generator which generates a gas flow. Ideally, the flow source 110 delivers flow at flow rates including between about 5 or 10 LPM and about 150 LPM. More preferably, the flow rates are between about 20 LPM and about 70 LPM. The flow rates may be between about 40 LPM and about 70 LPM. The range of flows delivered from flow source 110 to achieve sufficient patient oxygenation and CO₂ clearance as well as maintain a suitable patient pressure and/or a desirable expiratory resistance is typically between about 20 LPM and about 70 LPM. However, this range is dependent on the patient 300 being supported by the system 100, for example infants and children may not tolerate as high flow rates, and will require lower rates, as defined previously. Preferably for infants and children, the selected flow rate may be in a range of about 0.5 L LPM to about 25 LPM.

In some embodiments, the flow source 110 is configured to provide a continuous flow of gas at the selected flow rate. The continuous flow may be a unidirectional or positive net flow towards the patient. Furthermore, the selected flow rate for the flow source 110 may be a fixed flow rate. The fixed flow rate may be independent of the respiratory cycle of the patient 300.

In other embodiments, the flow source 110 may be configured to provide a flow of gas at a therapeutic flow rate that is time-varying (e.g. oscillating) and a controller controls a flow modulator to provide the therapeutic time-varying gas flow with an oscillating flow rate of: about 375 litres/min to about 0 litres/min, or preferably of about 240 litres/min to about 7.5 litres/min, or more preferably of about 120 litres/min to about 15 litres/min, and/or the oscillating flow rate has one or more frequencies of about 0.1 Hz to about 200 Hz, and preferably about 0.1 Hz to about 6 Hz, and more preferably about 0.5 Hz to about 4 Hz, and more preferably 0.6 Hz to 3 Hz. The gas flow modulator may comprise be the flow source 110 (where that could be a flow generator, O₂ source, ambient air or the like as previously discussed) and/or a valve or other device to modulate or otherwise vary parameters (e.g. flow rate, gas proportion) of a gas flow.

The oscillating flow rate may comprise a therapeutic flow rate component, wherein the therapeutic flow rate is about 375 litres/min to about 0 litres/min, or about 150 litres/min to about 0 litres/min, or is preferably about 120 litres/min to about litres/min, or is more preferably about 90 litres/min to about 30 litres/min. The oscillating flow rate may comprise a therapeutic gas flow component, wherein the constant (e.g. bias/base) flow rate component of the therapeutic gas flow is about 0.5 litres/min to about 70 litres/min. In some configurations, the therapeutic flow rate component is time-varying.

The oscillating flow rate may comprise a therapeutic flow rate component, wherein the therapeutic flow rate is about 0.2 litres/min per patient kilogram to about 2.5 litres/min per patient kilogram; and preferably is about 0.25 litres/min per patient kilogram to about 1.75 litres/min per patient kilogram; and more preferably is about 0.3 litres/min per patient kilogram to about 1.25 litres/min or about 1.5 litres/min per patient kilogram; and more preferably is about 0.4 litres/min per patient kilogram to about 0.8 litres/min per patient kilogram.

The one or more components of the time-varying (e.g. oscillating) gas flow may have one or more frequencies of about 0.3 Hz to about 4 Hz.

The oscillating flow rate may comprise at least one time-varying flow rate component, wherein each oscillating flow rate is about 0.05 litres/min per patient kilogram to 2 litres/min per patient kilogram; and preferably is about 0.05 litres/min per patient kilogram to about 0.5 litres/min per patient kilogram; and preferably about 0.12 litres/min per patient kilogram to about 0.4 litres/min per patient kilogram; and more preferably about 0.12 litres/min per patient kilogram to about 0.35 litres/min per patient kilogram. Alternatively, the oscillating flow rate may comprise at least one time-varying flow rate component, wherein each oscillating flow rate is in the range of 0.05 litres/min per patient kilogram to 2 litres/min per patient kilogram; and preferably in the range of 0.1 litres/min per patient kilogram to 1 litres/min per patient kilogram; and more preferably in the range of 0.2 litres/min per patient kilogram to 0.8 litres/min per patient kilogram.

The above are just examples, and other types of time-varying flow rates could be provided with the controller controlling a gas flow modulator to provide the time varying gas flow with the time-varying flow rate. The controller may have knowledge of the time-varying flow rate, and/or may measure the time-varying flow rate provided, e.g. by one or more flow sensors downstream of the flow modulator.

The system 100 may include an optional filter 170 positioned between the inspiratory conduit 160 and the patient interface conduit 180. A further optional filter 190 may be provided to treat expired gases exiting from the patient Gases flowing through the inspiratory conduit 160 are passed to the patient by way of the optional filter 170, the connector 200 and the invasive patient interface 120. The interface conduit 180 is connectable between the inlet port 220 of the connector 200 and the flow source 110 for providing fluid flow. In some embodiments, the interface conduit 180 may provide fluid coupling between the humidifier 140 and the inlet port 220 of connector 200.

In the context of the present disclosure, expired gases refers to gases that have been expired from the lungs. Gases exiting the patient's airway may or may not have been subjected to inhalation/exhalation as part of a spontaneous or non-spontaneous (i.e. artificially ventilated) respiratory cycle. Thus, gases exiting the patient's airway may include inspiratory gases which have not undergone alveolar gas exchange, old gases including those that have and have not been exchanged, as well as exchanged gases containing CO₂ as a result of alveolar gas exchange, irrespective of whether there has been inhalation/exhalation as part of a respiratory cycle.

In the context of the present disclosure, the inspiratory conduit 160 permits the provision of gases to the patient via the connector 200 (and optionally filter 170 and interface conduit 180) and invasive patient interface 120 for the provision of respiratory support. The respiratory support may include inhalation and/or exhalation as part of a spontaneous or non-spontaneous (i.e. artificially ventilated) respiratory cycle although that need not be the case. In many embodiments disclosed herein, respiratory gases conveyed by the inspiratory conduit 160 are provided as part of a respiratory support that are independent of cycles of spontaneous or non-spontaneous (i.e. artificially ventilated) inspiratory or expiratory breathing motion. As disclosed elsewhere herein, the respiratory support may comprise a flow of gases at a constant (albeit variable) flow rate, and/or a flow of gases that is time-varying or oscillatory.

In some embodiments, filters 170 and 190 may be non-removable and/or integral with conduit 160 and connector 200, respectively. Alternatively, the filters 170 and/or 190 may be releasably couplable with the conduit 160 and connector 200, respectively.

The inspiratory conduit 160 and patient interface conduit 180 may be one or more of corrugated, flexible, bendable, resistant to kink and/or heated (e.g., the conduits 160, 180 may include a heating element). Additionally/alternatively, the conduit 180 may be a breathable tube, such as described in U.S. Pat. No. 7,493,902 which is incorporated herein by reference.

In some embodiments, the system 100 as described herein may also include an optional pressure relief valve. In some embodiments, the system 100 as described herein may also include a sputum catcher. A sputum catcher may be beneficial when the connector 200 is used with a tracheostomy tube for the invasive patient interface 120.

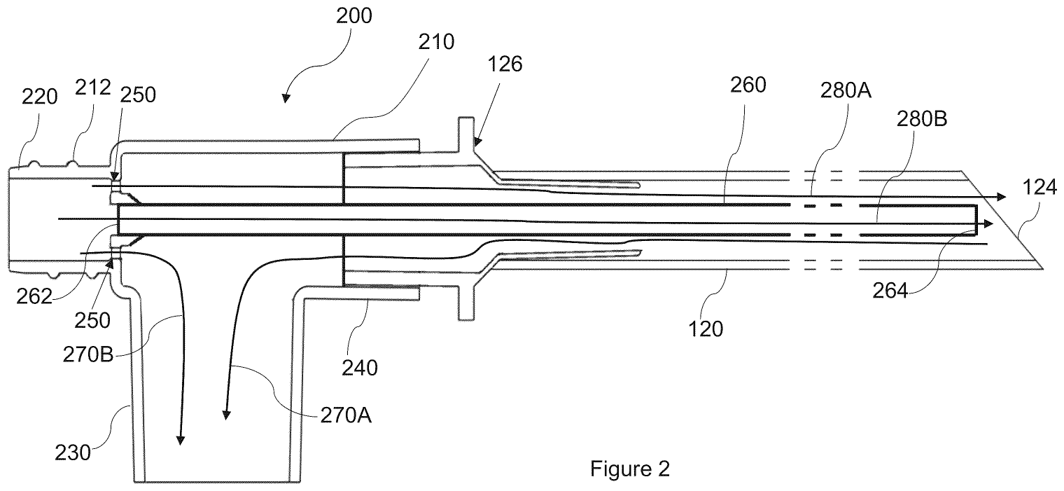

FIG. 2 provides a cross sectional view of a connector 200 for coupling with an invasive patient interface 120 providing respiratory support to a patient. The connector 200 includes a connector body 210 having an inlet port 220 couplable with a flow source 110 providing a flow of respiratory gas, a gases exit port 230 and a device port 240 couplable with the invasive patient interface 120 (shown as having indefinite length). An optional adapter 126 couples the invasive patient interface 120 and the device port 240 of the connector 200. Also provided is a lumen 260 having a first end 262 and a second end 264 which is disposed outside the device port 240. In this embodiment, the lumen first end 262 provides an inlet for receiving a flow of respiratory gases, and the lumen second end 264 provides an outlet disposed outside the device port 240 for delivery of the respiratory gas at a target location deeper in the patient's airway than the device port.

Connector body 210 defines a gas flow path 280 between the inlet port 220 and both the device port 240 and the lumen second end 264 enabling delivery of respiratory gas to the patient's airway via the invasive patient interface 120 (gas flow path 280A) and the lumen 260 (gas flow path 280B). Beneficially, connector body 210 defines gas flow path 280 in a manner which enables flow from a single flow source 110 to feed both the inspiratory pathway 280B defined by lumen 260 and the inspiratory pathway 280A. Alternatively, the inspiratory pathway 280A in lumen 260 may receive flow from a first flow source and the inspiratory pathway 280B in lumen 260 may receive flow from a second flow source that is different from the first flow source. The inspiratory pathway 280A is defined between the external wall of lumen 260 and the internal wall of invasive patient interface 120 and through which gases may flow. Connector body 210 also provides a gas outflow path 270A for outflow of mixed gases from the patient's airway which enter invasive patient interface 120 at proximal end 124. Connector body 210 also provides a gas outflow path 270B for outflow of gas from constrictions 250 via gases exit port 230. The bulk of flow through connector 200 is along gas outflow path 270B which, along with the degree of constriction of the gases exit port 230 contributes to establishing a patient airway pressure sufficient to deliver respiratory support according to embodiments of the disclosure.

Ideally, the lumen second end 264 is disposed, when in use, so that the target location is between device port 240 and proximal end 124 of the invasive patient interface 120 although that need not be the case and in some embodiments the target location may be outside the invasive patient interface. However, an arrangement in which the target location is between device port 240 and proximal end 124 of the invasive patient interface 120 prevents protrusion of the lumen second end 264 outside the invasive patient interface 120 and into the patient's airway. This safety feature may be provided to avoid contact between the lumen second end 264 and the airway wall, avoiding damage from e.g. perforation of the patient's airway (e.g. due to contact with the lumen tip or flows exiting the lumen) and reducing the risk of the lumen 260 becoming damaged and/or blocked with saliva or other fluids. However, if partial or complete blocking of lumen 260 does occur (e.g. if fluids enter the interface and the lumen), gas flow down the lumen 260 will instead be diverted around the lumen via gas flow path 280A and gases exit port 230 and the patient will still receive respiratory support. This arrangement may avoid the risk of barotrauma associated with systems in which there is only a single channel for delivery of respiratory gases and which may become blocked during use.

In some embodiments, the lumen 260 may comprise one or more small holes, slots or openings in the lumen wall toward the lumen second end 264. The one or more holes, slots or openings may provide additional outlets for the flow of gases which may be important in case of the main outlet opening at the lumen second end 264 becoming blocked with saliva or other fluids. Alternatively/additionally, the one or more holes, slots or openings may diffuse or disperse flows delivered to the patient via the lumen 260. This may improve CO₂ clearance e.g. by diffusing the flow within the lumen 260 into one or more jets upon exit through the one or more holes, slots or openings.

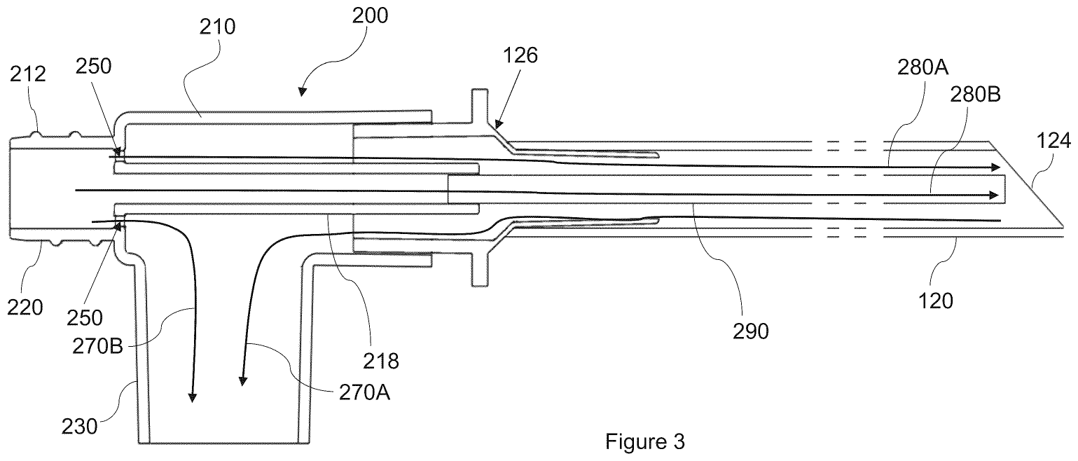

The entirety of lumen 260 may be integral with connector body 210 (e.g. FIG. 2), or part of lumen 260 may be integral with connector body 210 with the remainder of the lumen being provided by an elongate tube 290 (e.g. FIG. 3). In other embodiments still, the entirety of lumen 260 may be a separate component part such as an elongate tube which, when assembled together with connector body 210, forms connector 200. In some embodiments, the lumen 260 or part thereof may be defined by a catheter.

FIG. 3 is a sectional view of a modified connector 200 in which the connector body 210 defines guide portion 218 which, together with catheter 290 provides lumen 260. An advantage of this arrangement is that the lumen length can be trimmed by cutting the catheter to accommodate scenarios in which an adapter 126 is not used (and a shorter lumen length is required) to avoid lumen second end 264 from protruding out of proximal end 124 of invasive patient interface 120. Furthermore, since connector body 210 is typically formed of a substantially rigid material, and catheter 290 is a flexible lumen, manufacturing the connector 200 from component parts which are assembled together may simplify manufacture when compared with embodiments in which the connector body and lumen are manufactured as a unitary part. Guide portion 218 is shown as a channel into which catheter 290 may be inserted through device port 240 during assembly of connector 200. Thus, guide portion 218 also forms part of the lumen 260. It is to be understood however, that the guide portion may be embodied in a variety of ways including one or more of an eyelet, groove or slot with which catheter 290 may engage using e.g. friction fit, helical engagement, luer locking or other means to secure the structural elements that together form lumen 260. In some embodiments, guide portion 218 may be disposed on an internal wall of connector body 210, in the flow path from the inlet port 220 to the device port 240. That is, the structure of connector 200 need not locate lumen 260 concentrically with a flow path from the inlet port 220 to the device port; rather it may extend along and may follow the contours of an internal wall of the connector body 210.

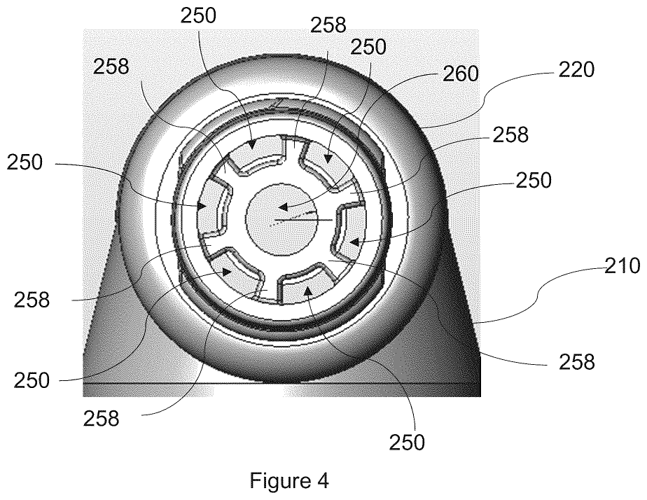

Ideally, connector 200 includes one or more constrictions 250 which are configured to generate a predetermined airway pressure when the connector is in use. FIG. 4 is an end view of connector 200 looking through inlet port 220 and shows one arrangement of constrictions 250. In this example, there are 6 constrictions 250 arranged circumferentially around lumen 260 which extends through connector body 210. The constrictions 250 may include regions of reduced cross-sectional area, relative to the inlet port 220, that accelerate the velocity of the gas flow before exiting mainly through gases exit port 230 or before being delivered to the patient via gas flow path 280A. The acceleration caused by constrictions 250 generates a predetermined airway pressure when the connector 200 is coupled with the invasive patient interface 120 and delivering flow from a flow source 110 into inlet port 220. As shown in the embodiment of FIGS. 2 and 3, the constrictions 250 are disposed between inlet port 220 and device port 240, upstream of where gas flow path 280A meets gas outflow path 270A.

In use, the flow of respiratory gas into the inlet port 220 is divided between at least the device port 240 and the lumen 260 and gases exit port 230, achieving a flow state in which airway pressure is maintained at the predetermined pressure generated by flow of gas through constrictions 250. Ideally, most flow exits the gases exit port 230 with a portion delivered to the airway by lumen 260 (which in some embodiments may be provided by a guide portion 218 together with catheter 290) and a portion may be delivered to the airway by invasive patient interface 120. The flow of respiratory gas in connector 200 maintains the desired predetermined pressure in the patient's airway. Typically, this pressure is in a range of about 0 cmH₂O to about 20 cmH₂O (preferably about 2 cmH₂O to about 20 cmH₂O, preferably about 2 cmH₂O to about 10 cmH₂O, more preferably about 2 cmH₂O to about 5 cmH₂O).

While FIG. 4 shows six constrictions 250, any number of constrictions may be deployed which, due to their total cross-sectional area achieve the desired effect of increasing the pressure in gas flow path 280 and in the patient's airway (relative to the pressure at gases exit port 230).

As would be apparent to one of skill in the art, the number, size and arrangement of constrictions 250 and diameter of lumen 260 in connector body 210 requires balanced selection of cross sectional areas for each constriction relative to the cross-sectional area of lumen 260 in order to achieve at least the predetermined patient pressure and at least the predetermined velocity of flow out of lumen 260, for a selected flow rate provided by flow source 110.

One objective of the embodiment shown in FIGS. 2 and 3 is to divide the flow of gas entering the inlet port 220 and flowing in gas flow path 280 between at least the device port 240 and the lumen 260, achieving a flow state in which respiratory gas is delivered to the target location at (at least) a predetermined velocity sufficient to achieve enhanced CO₂ clearance, particularly during provision of respiratory support for example in circumstances requiring respiratory stillness. For a given flow rate selected to provide the respiratory support, a larger inside diameter of lumen 260 will give rise to lower velocity flow at the lumen second end 264 and less gas mixing, hence the lumen must have dimensions carefully configured to achieve at least the desired predetermined velocity. In some embodiments, the desired predetermined velocity through the lumen second end is in a range of above about 0 m/s to about 25 m/s, such as a velocity in the range of about 5 m/s to about 20 m/s or more preferably about 10 to about 15 m/s, e.g. for a flow rate entering the inlet port 220 at flow rates of about 70 L/min. This may give rise to flow rates through the lumen second end of about 2 L/min to about 15 L/min at flow rates of about 70 L/min entering the inlet port 220. Velocities that can be achieved depend on the features and dimensions of the lumen and the connector body. These features may include but are not limited to cross-sectional area inside the lumen, cross-sectional area of the one or more constrictions forming the jet flow of gases, the length of the lumen and length of the one or more constrictions forming the jet flow of gases. Delivered flow rates may be affected by the flow source and/or features and/or dimensions of the lumen and/or connector body.

In some embodiments, lumen 260 may have an internal diameter which, relative to the internal diameter of the invasive patient interface 120, achieves at least the predetermined velocity. Internal diameters of about 2 to about 5 mm for lumen 260 may be suitable in embodiments utilizing an invasive patient interface 120 with an internal diameter of about 5 to 10 mm. In addition to selecting the internal diameter of lumen 260, it is important that the outside diameter of the lumen, relative to the internal diameter of the invasive patient interface 120 permits a flow of gas between the external surface of the lumen and the internal surface of the invasive patient interface. In some embodiments, a predetermined velocity exiting the lumen second end 264 of between about 5 m/s and about 60 m/s may be achieved. The length of lumen 260 may be similar to the length of invasive patient interface 120 since the lumen second end is, in some embodiments, intended to sit just inside the invasive patient interface proximal end 124. Thus, in embodiments utilising an ETT with a length of 150 mm to 400 mm, the length of lumen 260 may be about 150 mm to 400 mm or in some embodiments, about 0.5 mm or about 1 mm or about 1.5 mm shorter than the ETT length. In some embodiments, the lumen length is selected and/or the lumen is arranged relative to the connector such that lumen second end 264 terminates within the invasive patient interface (ETT) 120 set back about 0.5 to about 1.5 mm from the ETT proximal end 124.

FIG. 4 shows a plurality of constrictions 250 which are defined by support structures 258 that could also function as a guide portion for retaining a catheter 290 in an arrangement where the entire lumen 260 is not integrally formed with connector body 210. Thus, catheter 290 could be passed into the connector body 210 through the inlet port 220 or through device port 240, and engaged with the support structures 258 to retain the catheter in position during use. Engagement with the support structures may be by e.g. friction fit, helical engagement, luer locking or other means. It is to be understood that the constrictions exemplified in FIG. 4 may also be utilized in a connector embodiment in which lumen 260 provides an outflow path for mixed gases from the target location as described in relation to FIG. 5.

FIG. 5 provides a cross sectional view of an alternative embodiment of a connector 200 for coupling with an invasive patient interface 120 providing respiratory support to a patient. The connector 200 includes a connector body 210 having an inlet port 220 couplable with a flow source 110 providing a flow of respiratory gas, a gases exit port 230 and a device port 240 couplable with the invasive patient interface 120 (shown as having indefinite length). Also provided is a lumen 260 having a first end 262 and a second end 264 which is disposed outside the device port 240. In this embodiment, the lumen 260 provides an outflow path 270A for gases entering the lumen second end 264 at a target location deeper in the patient's airway than the device port 240. Thus, the connector of FIG. 5 may be regarded as an exit lumen connector. Lumen first end 262 is configured to provide for gases in the outflow path 270A to flow to the surrounding atmosphere. The connector body also provides for gases outflow through the device port 240 via outflow path 270B. Connector body 210 also defines a gas flow path 280A between the inlet port 220 and the device port 240 and a gas flow path 280B between the inlet port 220 and the gases exit port 230.

For similar reasons to those outlined in relation to the embodiment illustrated in FIGS. 2 and 3, lumen second end 264 is preferably disposed, when in use, so that the target location is between device port 240 and proximal end 124 of the invasive patient interface 120 although that need not be the case and in some embodiments the target location may be outside the invasive patient interface. However, it may be a safety feature of the connector for lumen second end 264 to be located just inside the opening of invasive patient interface 120 to avoid contact with the airway wall, avoiding damage from e.g. perforation and reducing the risk of the lumen 260 becoming blocked with saliva or other fluids. However, if partial or complete blocking of lumen 260 does occur (e.g. if fluids enter the interface and the lumen), gas outflow will be diverted around lumen 260 via gas flow path 270B and gases will still be removed from the patient's airway. This arrangement may avoid the risk of barotrauma associated with systems in which there is only a single channel for removal of respiratory gases and which may become blocked during use.

In some embodiments, the lumen 260 may comprise one or more small holes, slots or openings in the lumen wall toward the lumen second end 264. The one or more holes, slots or openings may provide additional inlets for the flow of gases which may be important in case of the main inlet opening at the lumen second end 264 becoming blocked with saliva or other fluids.

In the embodiment of FIG. 5, lumen 260 is intended to provide an outflow path 270A for exit of gases from the patient's airway. The connector 200 is configured to generate a predetermined airway pressure when coupled with the invasive patient interface 120 in use. In some embodiments this may be achieved by a constriction 250 (reduction in cross-sectional area) in the flow channel defined by the connector body 210 from inlet port 220 as shown. A conditioning portion 254 may also be provided to condition the flow into the invasive patient interface 120. Conditioning the flow may provide laminarising the flow in the embodiment of FIG. 5 by virtue of a sufficient length of the straight portion of conditioning portion 254 having constant diameter as shown. In some embodiments it may be desirable for the internal geometry of the connector body 210 to gradually taper into the conditioning portion 254 since abrupt changes in the internal geometry can give rise to turbulence. Alternatively/additionally, the conditioning portion or other internal geometry of the connector body 210 may condition the flow by increasing turbulence or otherwise modifying the flow characteristics. For these embodiments (not shown), the conditioning portion 254 or connector body 210 may comprise internal wall features such as a helical ridge, protrusions or other surface roughening features.

Gases near the target location enter the second end 264 of lumen 260 and flow in the direction of a downward pressure gradient to e.g. atmosphere via a gas outflow path defined by the lumen. This provides a mechanism for removal of mixed gases in the patient's airway (including e.g. fresh, old and expired gases) and contributes to clearance of CO₂. CO₂ clearance may be improved by increasing the pressure gradient. In some embodiments, a pressure gradient of from about 20 cmH₂O to above 0 cmH₂O, preferably about 10 cmH₂O to above 0 cmH₂O may be desired and in some embodiments, a pressure gradient of about 5 cmH₂O may be preferred. Connector 200 utilises the predetermined airway pressure generated by the connector in combination with the arrangement of lumen 260 with its second end 264 at the target location to achieve a flow state in which there is out flow through lumen 260 at a velocity which is sufficient to achieve enhanced CO₂ clearance, relative to a similar connector not having lumen 260. CO₂ clearance may also be increased by increasing the cross sectional area (or reducing the resistance to flow) of the gas outflow path defined by the lumen.

In some embodiments, the pressure gradient and hence the flow rate and optionally the velocity at which gases exit lumen 260 can be increased, thereby increasing CO₂ clearance, by use of a negative pressure source or vacuum (not shown) in fluid communication with lumen first end 262. A negative pressure source may actively draw out gases creating greater CO₂ clearance than when lumen 260 is open to atmosphere (for a given patient pressure). A controller may operate the negative pressure source and the flow source to balance flow (and/or pressure) delivery against the rate of gas extraction from lumen 260. This may involve increasing or decreasing the level of suction thereby increasing or decreasing the pressure gradient and hence the flow rate and optionally the velocity of gases flowing through the lumen.

As described in the context of the embodiments shown in FIGS. 2 and 3, in the exit lumen connector of FIG. 5 the entirety of lumen 260 may be integral with connector body 210, or part of the lumen may be integral with connector body 210 with the remainder of the lumen being provided by an elongate tube 290 as shown. In other embodiments still, the entirety of lumen 260 may be a separate component part such as an elongate tube which, when assembled together with connector body 210, forms connector 200. In some embodiments, the lumen 260 or part thereof may be defined by a catheter.

In the embodiment shown in FIG. 5, connector body 210 may be

regarded as defining a guide portion 218 which, together with catheter 290, provides lumen 260. An advantage of this arrangement is that the lumen length can be trimmed by cutting the catheter to accommodate scenarios in which an adapter 126 is not used (and a shorter lumen length is required) to avoid lumen second end 264 from protruding out of proximal end 124 of invasive patient interface 120. Furthermore, since connector body 210 is typically formed of a substantially rigid material, and catheter 290 is a flexible lumen, manufacturing the connector 200 from component parts which are assembled together may simplify manufacture when compared with embodiments in which the connector body and lumen are manufactured as a unitary part. Guide portion 218 is shown as a channel into which catheter 290 may be inserted from outside of the connector body 210, during assembly of connector 200. Catheter 290 may also be inserted over an exterior surface of guide portion 218. Thus, guide portion 218 also forms part of the lumen 260. It is to be understood however, that the guide portion may be embodied in a variety of ways including one or more of an eyelet, groove or slot with which catheter 290 may engage using e.g. friction fit, helical engagement, luer locking or other means to secure the structural elements that together form lumen 260. In some embodiments, guide portion 218 may be disposed on an internal wall of connector body 210, in the flow path from the inlet port to the device port. In other embodiments, such as the one shown in FIG. 5, guide portion 218 may be molded into the connector body 210 in a manner which provides for lumen first end 262 to open to atmosphere. In any case, the structure of connector 200 need not locate lumen 260 concentrically with a flow path from the inlet port 220 to the device port; rather it may extend along and may follow the contours of an internal wall of the connector body 210.

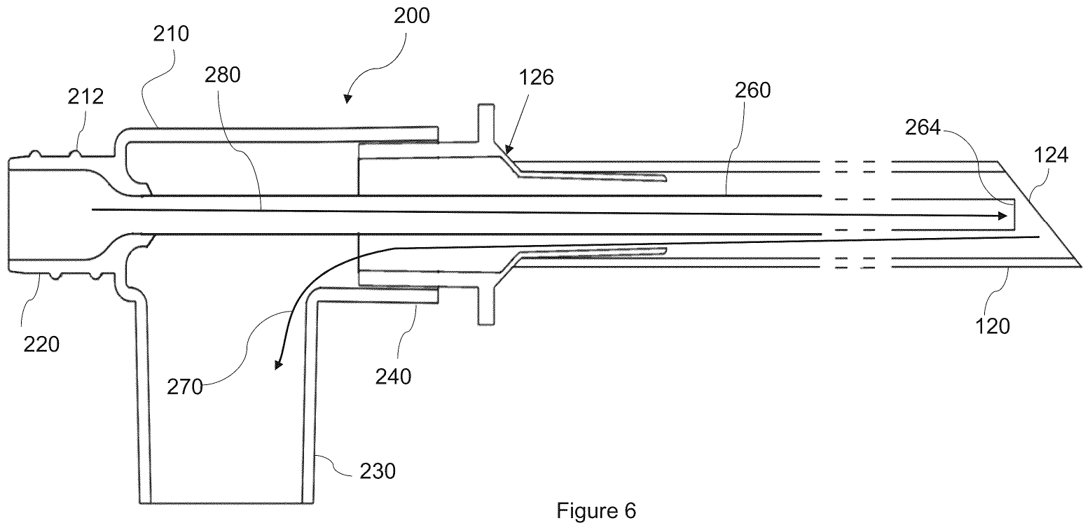

FIG. 6 provides a cross sectional view of an alternative embodiment of a connector 200 for use in a system for providing respiratory support to a patient using an invasive patient interface 120. System 100 includes a flow source 110 providing a flow of respiratory gas. The connector 200 has an inlet port 220 receiving a flow of respiratory gas from flow source 110, a device port 240 for coupling with the invasive patient interface 120, and a gases exit port 230. A lumen 260 is provided with a lumen second end 264 configured to be disposed at a target location inside the invasive patient interface.

Connector 200 defines a first gas flow path 280 for delivering all of the flow of respiratory gas into the lumen to the target location. Additionally, connector 200 defines an exit flow path 270 for escape of gases exiting the airway through the device port and out of gases exit port 230. The flow of respiratory gas through the lumen second end 264 is determined by the system 100 to be at least a predetermined velocity (achieved by lumen 260 providing the constriction), giving rise to gas mixing near the target location and increased removal of CO₂ through the invasive patient interface 120 which provides exit flow path 270 through gases exit port 230. The respiratory support provided by the system 100 may maintain a period of safe respiratory stillness.

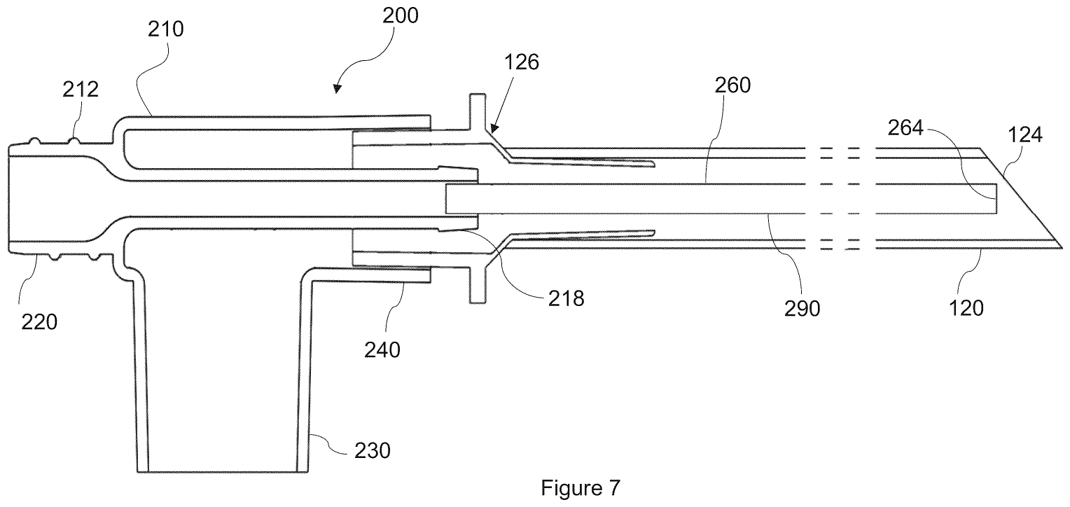

The entirety of lumen 260 may be integral with connector body 210 as in FIG. 6, or part of lumen 260 may be integral with connector body 210 with the remainder of the lumen being provided by an elongate tube 290. In other embodiments still, the entirety of lumen 260 may be a separate component part such as a catheter which, when assembled together with connector body 210, forms connector 200. Assembly of separate component parts may utilize guide portions as disclosed in the context of other embodiments in other Figures and it is to be understood that the features of those guide portions are also contemplated and expressly stated to be compatible with the connector in FIG. 6. For example, FIG. 7 is a sectional view of a modified version of the connector 200 of FIG. 6, in which connector body 210 defines guide portion 218 which, together with catheter 290 provides lumen 260.

The period of safe apnoea achievable according to use of the connector 200 of FIGS. 6 and 7 is at least in part due to the desirable velocity of flow delivered through the lumen which causes gas mixing in the vicinity of the target location giving rise to increased CO₂ clearance. Ideally, the target location is within the invasive patient interface 120 to limit the risk of the lumen becoming occluded and preventing flow of respiratory gas form the flow source to the patient's airway since, in the embodiment shown in FIGS. 6 and 7, all flow from the flow source is directed to the first gas flow path within lumen 260 and delivered to the target location. Ideally, the target location is close to the proximal end of the invasive patient interface to maximise the gas mixing effect deep in the patient's airway such as near the patient's carina or deeper in the patient's bifurcated airway. It is contemplated, however, that in other embodiments the connector body may define a further flow path for delivery of a portion of the flow of respiratory gas from the flow source to the invasive patient interface, more akin to the embodiments exemplified in FIGS. 2 and 3.

Embodiments of various connectors described herein may include an adapter 126 coupling the invasive patient interface 120 and the device port 240 of the connector 200, as per the Figures. However, the adapter 126 may be omitted and the device port 240 and invasive patient interface 120 may instead be directly coupled, as would be appreciated by a person skilled in the art. As such, it is to be understood that the present disclosure is not to be limited to require an adapter 126 for coupling with the invasive respiratory device 120.

The embodiments of FIGS. 6 and 7 do not show constrictions 250 configured to receive a flow of gas. However, it is to be understood that in these and all embodiments disclosed herein, a predetermined patient pressure may be achieved within the patient's airway by altering the total cross-sectional area of the gases exit port and/or the cross-sectional area of the lumen 260 to achieve a pressure loss between the device port 240 (or lumen second end 264) and gases exit port 230 which is equivalent to about the predetermined patient pressure of greater than 0 cmH₂O. The generated patient pressure may be between about 2 cmH₂O and about 20 cmH₂O, or about 2 cmH₂O to about 10 cmH₂O, or about 2 cmH₂O and about 5 cmH₂O, or about 5 cmH₂O to about 10 cmH₂O. This may be achieved by e.g. applying a filter to the gases exit port. It is to be understood that this approach to modifying or tuning the patient pressure may be used as an alternative or in addition to constrictions configured to generate desired patient pressures.

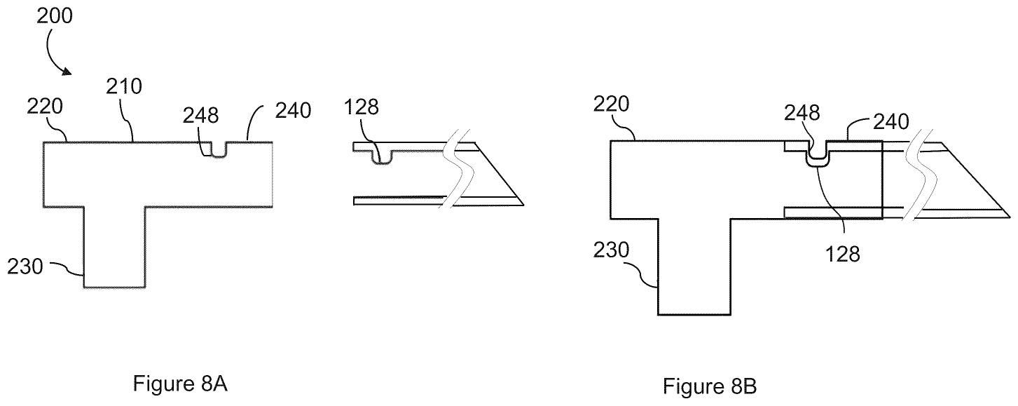

Embodiments of the various connectors 200 described herein and shown in the Figures may include a locating feature which prevents the lumen second end 264 from protruding out of the invasive patient interface 120 when in use, as shown in FIGS. 8A and 8B. In some embodiments, the locating feature is configured to maintain a desired setback of the lumen second end 264 from proximal end 124 of the invasive patient interface 120 when coupled with the device port 240 either directly or indirectly using adapter 126. In one example, a locating feature 248 may be formed in connector body 210, e.g. on an internal wall of the connector body just inside of device port 240. The locating feature 248 may include an engagement structure for releasably coupling the device port 240 with a corresponding structure on the invasive respiratory device 120 or an adapter 126 connected to the invasive respiratory device 120. The engagement structure may include one or more of a protrusion, a rib, a groove and a flange on an internal or external surface or wall of connector body 210 at device port 240.

FIGS. 8A and 8B, are schematic illustrations showing sectional views of a connector 200 with a locating feature in the form of a protrusion 248 formed on an internal wall of connector body 210. The protrusion 248 cooperates with a corresponding locating feature in the form of a notch 128 in adapter 126 or invasive patient interface 120. When the adapter or invasive patient interface is inserted into the device port 240 as shown in FIG. 8B, notch 128 on adapter 126 receives and engages with protrusion 248 on connector 200. The engagement may provide releasable or non-releasable coupling of connector 200 and adapter 126/invasive patient interface 120. Once engaged, the location of lumen second end 264 is at a predetermined set back distance from the invasive patient interface proximal end 124. This provides a safety feature that is viable for all embodiments of the connectors disclosed herein, providing at least a degree of protection against perforation of the patient's airway by the lumen second end 264, and against blockage of or damage to the lumen itself, and causing localised tissue damage or barotrauma in the patient. Although a protrusion 248 and a notch 128 is illustrated, a person skilled in the art would appreciate that the locating features and engagement structure could take any form that provides for releasable or non-releasable coupling between the respective components.

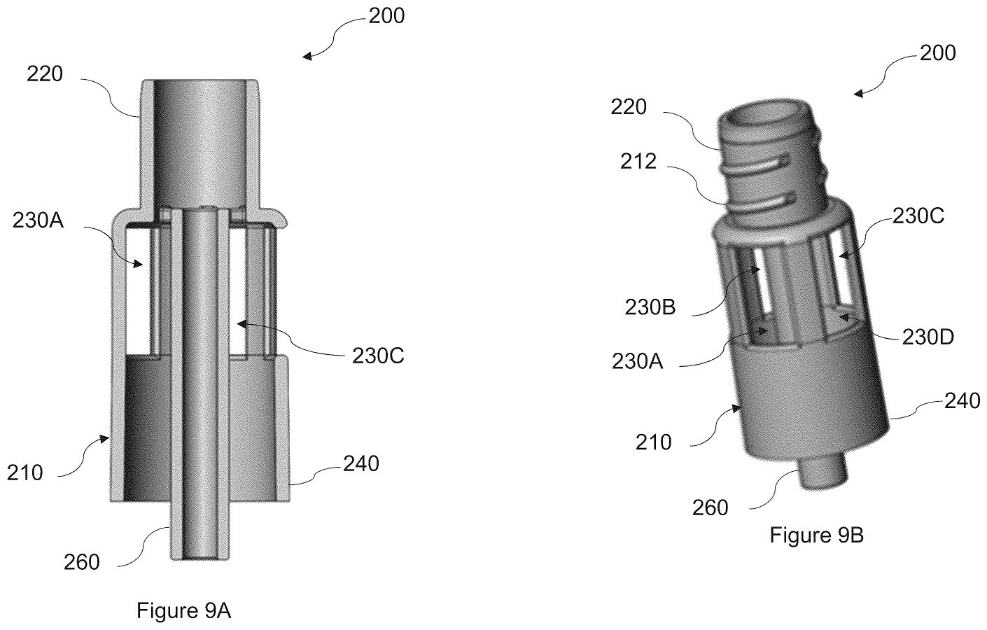

FIGS. 9A and 9B are sectional and perspective views respectively of an alternative connector 200 in which gases exit port 230 is provided by multiple openings 230A, B,C,D. FIGS. 9A and 9B show connector body 210 with a helical thread 212 for coupling connector 200 with a corresponding thread provided on conduit 180 (FIG. 1) which couples flow source 110 with connector 200. It is to be understood, however, that other coupling means such as luer lock, friction fit and the like may be utilized to achieve releasable coupling between the connector inlet port 220 and conduit 180. In FIGS. 9A and 9B, device port 240 and lumen 260 are arranged in a manner similar to the arrangement of FIGS. 2, 3, 5, 6 and 7. Advantages associated with provision of multiple openings 230A, B,C,D may include mitigating risk of clinicians or other users accidentally blocking the gases exit port 230 when provided as a single opening. Complete closure of the gases exit port could lead to barotrauma in the patient.

Embodiments of the various connectors 200 described herein and shown in the Figures may include filter 190 couplable with the gases exit port 230 of the connector 200 for filtering the gases from the connector body 210, as shown in FIG. 1. Expiratory flow from the patient 300 passes along the gas outflow path 270 and exits connector 200 via the gases exit port 230 to atmosphere. A filter 190 may be used if the patient 300 is infectious or is provided with gases containing nebulized drugs that can be harmful to surrounding personnel or to the environment. The filter 190 preferably captures contaminants, aerosols, pathogens, etc. in the gases exiting the gases exit port 230. The filter may be annular (e.g. in the case of FIGS. 9A and 9B) or it may be an in-line filter (e.g. in the case of FIGS. 2, 3, 5, 6 and 7).

Alternatively/additionally, one or more connectors of the present disclosure may be provided with a variable aperture for adjusting resistance to flow of gases exiting the connector through the gases exit port 230. The features providing the adjustable aperture may be formed in the main connector body 210 or in a connector body extension (not shown) which couples with the exit port 230 e.g. by friction fit or threaded coupling. In use, the variable aperture may be used to control resistance to flow of gases exiting through the gases exit port 230 (or extended gases port) which in turn gives rise to different patient pressures achieved within the patient during provision of respiratory support.

Adjusting resistance to flow of gases exiting the gases exit port 230 may be useful in some embodiments. For example, increasing resistance to flow through the gases exit port 230 by reducing the size of the variable aperture may increase patient pressure which in turn, may increase CO₂ clearance for connectors disclosed herein that utilize a lumen 260 for delivery of respiratory gas to the target location in the patient's airway. Alternatively/additionally, increasing resistance to flow by decreasing the size of the variable aperture may be desirable to increase airway pressure or Positive End Expiratory Pressure (PEEP).

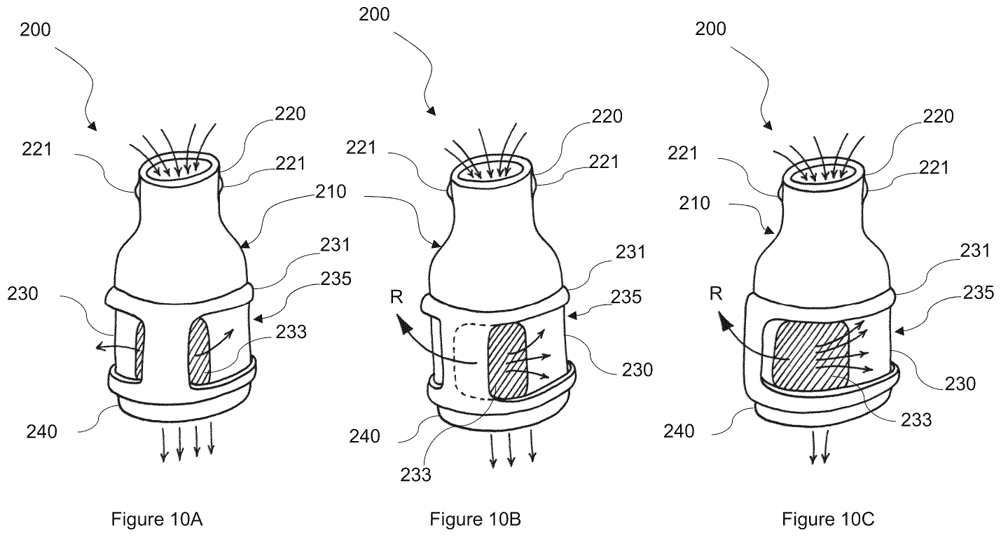

In one example according to FIGS. 10A to 10C, connector body 210 has similar features to connectors described elsewhere herein including gases port 220 which receives respiratory gases from a flow source via a conduit (not shown) which may be coupled with the connector using e.g. friction fit projections 221, and device port 240 configured to couple with an invasive patient interface 120 (not shown). Also not shown for simplicity is a lumen 260 which may be provided with the connector as described previously. Gases exit connector body 210 through gases exit port 230 which has a variable aperture defined by first opening 233, which is provided in a wall portion defining the gases exit port 230, and a moveable collar 231. Collar 231 is arranged around at least part of the wall portion defining the exit gases port 230. The hashed region represents first opening 233 and the stylistic arrows represent the direction of flow of gases when in use. The collar 231 may be a complete annulus or an incomplete annulus such as a broken ring or collar. The collar 231 has a second opening 235. Movement of collar 231 adjusts an amount of overlap between the first opening 233 and the second opening 235 to vary the extent to which the variable aperture, through which gases exit connector 200, is open. When collar 231 is rotated so that the solid part of the collar is arranged over first opening 233, the variable aperture is at its smallest opening size providing maximum resistance to flow to gases exiting the connector through exit port 230. When collar 231 is rotated so that there is less overlap between first opening 233 and second opening 235, the variable aperture is larger, reducing the resistance to flow.

FIGS. 10A to 10C show collar 231 rotated to different positions which provide different variable aperture configurations. FIG. 10A shows two small apertures providing maximum resistance to flow available with this collar arrangement. It is to be noted that the embodiment shown provides a safety feature in that the collar structure 231 does not allow the first opening 233 of the gases exit port 230 to be fully covered. In FIG. 10B collar 231 has been rotated clockwise relative to FIG. 10A as shown by arrow R, such that part of the solid section of collar 231 is occluding first opening 233 which reduces the resistance to flow of gases exiting the gases exit port 230 relative to FIG. 10A. In FIG. 10C collar 231 has been further rotated clockwise in the direction of arrow R such that the variable aperture in the most open position providing the lowest resistance to flow of gases exiting the gases exit port 230. It is to be understood that reducing the resistance to flow of gases exiting the gases exit port 230 reduces the total resistance to flow of the connector 200, which is represented as the pressure loss between the gases port 220 which receives gases from the flow source, and the gases exit port 230. Therefore, assuming other connector and flow parameters remain the same, increasing the size of the variable aperture reduces the resistance to flow which in turn reduces the patient pressure. Conversely, assuming other connector and flow parameters remain the same, decreasing the size of the variable aperture increases the resistance to flow which in turn increases the patient pressure.

Although FIGS. 10A to 10C show collar 231 being rotationally movable relative to the wall portion of the gases exit port 230, it is to be understood that a ring or other annular-type collar may be translated along the wall portion to achieve a varying aperture size. Furthermore, it is to be understood that the second opening 235 which is formed in the collar 231 may take the form of a single opening of any shape or size that can be manufactured into the collar, or a plurality of openings or holes which may be variably aligned with the first opening by sliding the collar 231 along or around the gases exit port 230. In some embodiments, the connector 200 may provide a visible and/or tactile indicator providing a guide to the user as to different patient pressures that may be achieved for a given flow rate (e.g. 70 L/min) according to the position or degree of openness of the variable aperture. For example, the connector 200 may have a plurality of discrete markings on the outer wall of the exit gases port 230 corresponding to e.g. 2 cmH₂O, 5 cmH₂O, 10 cmH₂O, 15 cmH₂O and 20 cmH_{2O} and which, when aligned with a marking on the collar 231, indicate that patient pressure deliverable for the corresponding aperture size at a given flow rate. It is to be understood that the discrete values provided in this example are not limiting on the number, range or step size of discrete patient pressures that may be indicated.

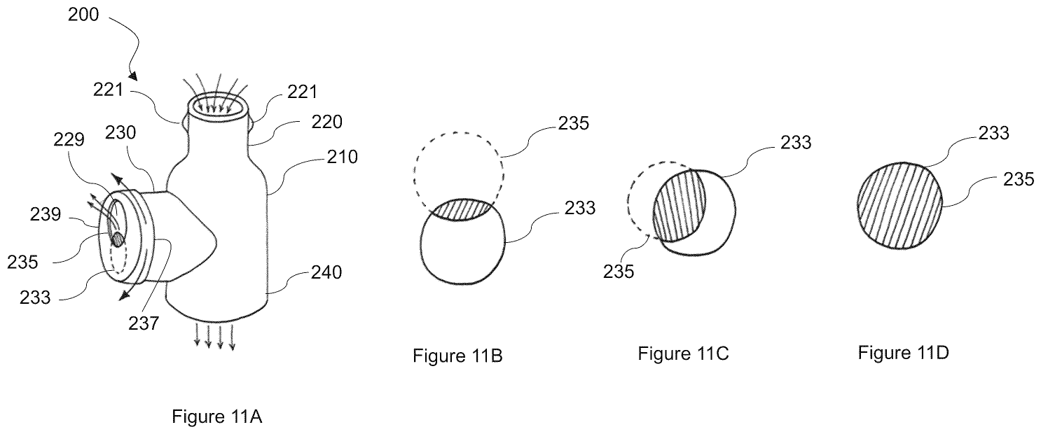

In another example according to FIGS. 11A to 11D, connector body 210 has similar features to connectors described elsewhere herein including gases port 220 which receives respiratory gases from a flow source (not shown) and device port 240 configured to couple with an invasive patient interface 120 (not shown). Also not shown for simplicity is a lumen 260 which may be provided with the connector as described previously. In the embodiment shown, the gases exit port 230 is a side port of connector 200 which provides a structure to which cap 237 may be applied to provide a variable aperture. In some embodiments the cap is permanently formed over the opening of exit gases side port 230 and in other embodiments, the cap is removably applied by helical thread, friction fit or the like. Cap 237 has a first member 229 with a first opening 233 (represented by a hashed region) and a second member 239 with a second opening 235. One of the first and second members is moveable relative to the other member to alter the degree of overlap between the first and second openings 233, 235 in those members. As can be seen in FIG. 11A, the first and second members are circular discs with respective openings offset from center. Relative rotational movement between the first member 229 and second member 239 varies an amount of overlap between the first and second openings 233, 235 to define and alter the variable aperture. The stylistic arrows represent the direction of flow of gases.

In FIG. 11A, first member 229 is almost entirely concealed beneath second member 239 (except for the part that can be seen through second opening 235). In some embodiments, first member 229 be permanently applied over the opening of exit gases side port 230, or fixed in place as part of a cap 237 which is applied over the opening of the exit gases side port as described previously. Thus first member 229 is typically stationery in use, with the second member 239 rotationally moveable relative to the first member.

FIGS. 11B to 11D are schematic illustrations showing relative movement of second opening 235 (represented by broken lines) relative to stationery first opening 233. The first and second members 229, 239 in which the openings are provided have been omitted for simplicity. The variable aperture formed by the overlapping openings is represented by hashed lines. In FIG. 11B, there is a small overlap between the first and second openings 233, 235 providing a small aperture and relatively high resistance to flow of gases exiting the gases exit side port 230. In FIG. 11C, there is a larger overlap between the first and second openings 233, 235, providing a larger aperture and moderate resistance to flow of gases exiting the gases exit side port 230. In FIG. 11D, the first and second openings 233, 235 overlap in their entirety providing the maximum available aperture size and lowest resistance to flow of gases exiting the gases exit side port 230. It is to be noted that the embodiment shown provides a safety feature in that the arrangement of the first and second openings 233, 235 in first and second members 229, 239 does not allow the gases exit side port 230 to be fully closed. Similar to the embodiment of FIGS. 10A to 10C, the connector 200 may provide a visible and/or tactile indicator providing a guide to the user as to different patient pressures that may be achieved for a given flow rate (e.g. 70 L/min) according to the position or degree of openness of the variable aperture.

Embodiments of the various connectors 200 described above and shown in the Figures may include one or more gas sampling ports (not shown) for sampling one or more characteristics of gases in the connector and/or the invasive patient interface and/or the patient airway. The one or more characteristics of the gases may include pressure, flow rate, velocity, concentration, gas constituents, temperature, humidity, contaminants, aerosols and/or pathogens. The one or more gas sampling ports may be located to sample gases inside connector body 210 and/or at or near one or more of the gases exit port 230, the device port 240, inside the lumen 260 and at the lumen first end 262 or lumen second end 264.

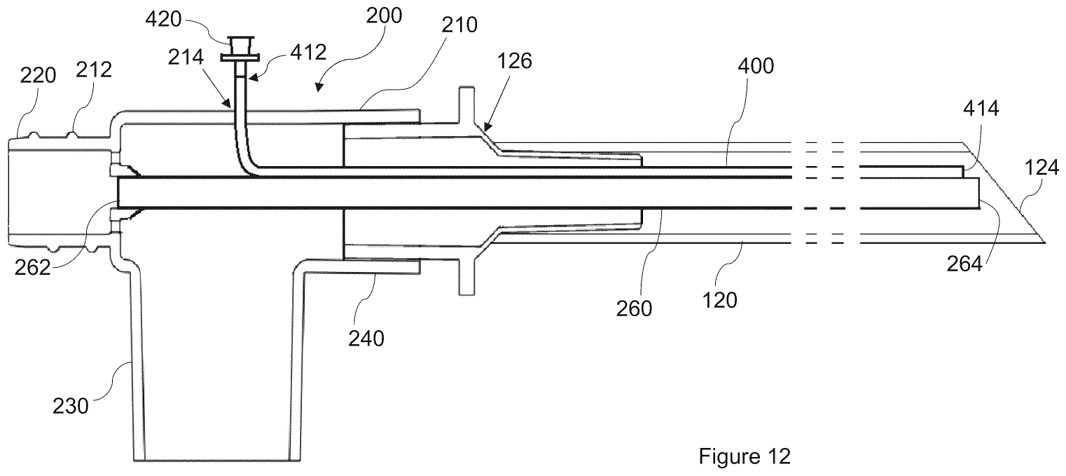

FIG. 12 provides an example of a connector 200 with a gases sampling port 214 according to an embodiment of the disclosure which utilises the connector of FIG. 2 although it is to be understood that similar features may be incorporated to achieve gas sampling in respect of other connectors disclosed herein. The gases sampling port 214 is configured to facilitate fluid coupling of sampled gases from a sampling location within the patient's airway to an instrument for analysing the sampled gas. Gases of particular interest include O₂ and CO₂ which may be analysed by an instrument such as an oxygen analyser or capnography machine (not shown). Monitoring CO₂ using gases sampling port 214 may provide utility in ascertaining quantities or concentrations of CO₂ cleared from the target location within the patient's airway, which may provide a real-time quantitative measure of the amount of CO₂ (lungs and/or blood) in the patient. Monitoring O₂ using gases sampling port 214 may provide utility in ascertaining oxygenation of the patient.

In the embodiment shown, fluid communication to the analysing instrument is provided by a gases sampling line 400 having a sampling end 414 which is locatable within the invasive patient interface 120 for sampling gases at a sampling location within the patient's airway. In some embodiments a separate gas flow conduit may be provided between the gases sampling line and the analysing instrument as described below. Ideally, the sampling location is near the patient's carina to obtain an accurate measure of gas composition, concentration or the like deep within the patient's airway. This may be used to determine the extent to which CO₂ or O₂ has built up in the patient's lungs and airways and may be used to influence decisions about the type of respiratory support that may be required for the patient.

In the example shown in FIG. 12, sampling end 414 is located just distally (i.e. set back slightly) from the lumen second end 264. This may provide better performance than a sampling end 414 having an opening which is in line with the opening of lumen second end 264 since the set-back reduces the risk of the sampling end becoming blocked e.g. by fluid. Additionally, sampling end 414 being located just distally from the lumen second end 264 may provide more accurate CO₂ readings than a sampling end 414 having an opening which is in line with the opening of lumen second end 264. However, it is to be understood that the set-back of sampling end 414 may not be necessary and in some embodiments, the sampling end may be located proximally (i.e. deeper in the patient's airway) from lumen second end 264. In some cases, sampling end 414 may protrude outside of invasive patient interface 120 however in most cases this is not desirable as it increases risk of the gases sampling line 400 becoming blocked with fluids or contacting (and potentially damaging) the airway wall.

It is to be understood that gases sampling port 214 may be utilised to sample gases such as CO₂ from elsewhere such as e.g. within the connector body 210 using a catheter with a sampling end 414 located within the connector body (or elsewhere) or by sampling from the gases sampling port 214 using a suitable gas flow conduit (not shown) connecting the sampling port to the analysis instrument. Thus in some embodiments, the gases sampling port 214, or a gases sampling line 400 inserted through the gas sampling port 214 may be couplable with a gas flow conduit using a suitable connector as described below.

The gases sampling line 400 may take any suitable form. In one example, at least part of the gases sampling line 400 may be formed integrally with the connector lumen 260. Thus, the connector 200 may provide e.g. a dual lumen catheter or extrusion which provides gas flow functionality for both the lumen 260 which deliver gases deep within the patient's airway, and the gases sampling line which provides for sampling of gases from a similar airway location for detection of e.g. CO₂. In other embodiments, the gases sampling line 400 may be provided by a separate catheter or extrusion which is assembled into the connector 200.

In one example, the connector of FIG. 12 may be assembled by passing the gases sampling line 400 through gases sampling port 214. In some embodiments, the gases sampling line 400 may be insertable or removable from connector 200 during use. One or more guide portions (not shown) may be provided within the connector to guide insertion of the gases sampling line 400 into the device port. Alternatively/additionally, one or more clips, fasteners, eyelets or the like may be utilised to couple the gases sampling line with the lumen 260 although this need not be the case. FIG. 12 shows gases sampling line 400 with connecting end 412 terminating in a connector 420. Connector 420 may be e.g. a luer or other connector suitable for providing fluid coupling between the gases sampling line 400 and a gas flow conduit conveying the sampled gas to the analysis instrument. It is to be understood, however, that gases sampling line 400 need not protrude outside the connector 200, and the connector 420 may be assembled into or form part of the connector body 210.

Embodiments of the present disclosure provide improvements in CO₂ clearance during delivery of respiratory support. In particular, the present disclosure provides benefits in systems delivering respiratory support while the patient is apnoeic and in a state of respiratory stillness, in which there is little or no movement of the patient's abdomen and thorax, or at least in which the respiratory support being delivered causes little or no movement of the patient. Embodiments of the present disclosure therefore contribute to maintaining a safe prolonged period (or periods) of respiratory stillness. Additionally, the inventors have discovered that providing a flow of respiratory gases at higher velocity and ideally, at certain patient pressures leads to increased gas mixing and have developed devices that take advantage of this to provide improved CO₂ clearance during respiratory support. The benefit may be best realised when gas mixing is deep in the patient's airway, such as e.g. close to the patient's carina or deeper in the patient's bifurcated airway. Thus, embodiments disclosed herein provide a connector for use with an invasive patient interface which includes a lumen, such as a catheter, for reducing the dead space of the flow path comprising the patient's airway, and providing and/or removing a flow of gases to increase gas mixing and CO₂ clearance.

It is to be understood that several factors contribute to enhanced CO₂ clearance in various embodiments and aspects of the disclosure and these factors can interrelate. One factor is the velocity of gases at a target location within the patient's airway. Increasing the velocity of gases at a target location within the patient's airway increases CO₂ clearance. Within a patient's airway includes within the invasive patient interface, at an end of the invasive patient interface, and outside of the invasive patient interface but within the patient's airway (for example between the end of the invasive patient interface and the patient's carina). In some configurations, the target location is at or close to the carina. The velocity of gases at the target location may be based on one or more exit velocities (e.g. average velocity) of gases exiting the lumen second end for delivery to the target location, and the distance of the lumen second end from the target location (e.g. the carina). The exit velocity of gases exiting the lumen second end may be influenced by factors such as flow rates at which gases are provided to the inlet port, the resistance to flow of the flow path in which gases flow to the patient (e.g. cross sectional area of the flow path and/or the geometry of the internal walls of the lumen defining the flow path and/or the length of the flow path, and/or the presence or absence of constrictions in the flow path), the pressure gradient across which the gases flow, the flow rate of gases exiting the gases exit port, the velocity of gases exiting the gases exit port and the resistance to flow through the gases exit port. In some embodiments, the velocity of gases exiting the gases exit port may be influenced by the exit velocity of gases exiting the lumen second end. In some scenarios, a desirable exit velocity of gases exiting the lumen second end is achieved about 5 mm from the carina since there is a risk of damage if gases are delivered too close to or at the carina. In most medical procedures however, a clinician locates the proximal end of the invasive patient interface (e.g. ETT) about 4 cm from the carina which limits how close the lumen second end is to the carina. Suitable exit velocities may include e.g. a velocity in a range of above about 0 m/s to about 25 m/s at a selected flow rate of about 5 L/min to about 70 L/min of the flow of gas provided to the lumen, such as a velocity in the range of about 5 m/s to about 20 m/s. In some embodiments, the exit velocity is a velocity in a range of about 10 m/s to about 15 m/s.

Another factor that contributes to enhanced CO₂ clearance in various embodiments and aspects of the disclosure is the flow rate of gases exiting the patient. In some configurations, this flow rate may be the flow rate of gases exiting via the gases exit port. Increasing this flow rate of gases exiting the patient increases CO₂ clearance. The flow rate at which gases exit the patient is related to the flow rate at which gases are provided to the patient, for example through the lumen second end. The resistance to flow within the connector and/or delivery lumen (e.g. lumen 260) may affect the flow rate delivered to the patient. In some embodiments, for a given driving pressure of the flow source, altering the resistance to flow in the connector and/or delivery lumen may alter the flow rate being delivered to the patient and hence alter the flow rate exiting the patient via the gas outflow path. Resistance to flow can be influenced by parameters of the flow path in which gases flow to the patient (e.g. cross sectional area of the flow path and/or the geometry of the internal walls of the lumen defining the flow path and/or the length of the flow path). As indicated above, a flow rate of gases provided by the flow source may be in a range of 0 to about 150 LPM, preferably about 20 LPM to about 70 LPM. In some embodiments, the flow rate of gas provided by the flow source is at least about 40 LPM and in many applications is about 70 LPM. Part of or all of the flow rate of gases provided by the flow source may be provided to the patient (via the lumen). In some configurations, the flow rate of gases exiting the patient is inversely related to the resistance to flow of the exit flow path from the patient to atmosphere, for example, the higher the resistance to flow, the lower the flow rate. This resistance to flow can be influenced by parameters of the exit flow path from the patient to atmosphere (e.g. cross sectional area of the flow path and/or the geometry of the internal walls of the lumen defining the flow path and/or the length of the flow path). The resistance to flow of the exit flow path from the patient to atmosphere is related to pressures delivered to the patient, for example the greater the resistance to flow in the exit flow path, the greater the delivered pressures.

Another factor that contributes to enhanced CO₂ clearance in various embodiments and aspects of the disclosure is the pressure differential between inside the patient's airway and atmosphere. A pressure differential can affect flow rate of gases exiting the patient's airway, where a greater pressure gradient will give rise to a greater flow rate of gases exiting the patient's airway. The pressure differential between inside the patient's airway and atmosphere may be influenced by factors such as flow rates at which gases are provided to the inlet port, the resistance to flow of the flow path in which gases flow to the patient (e.g. cross sectional area of the flow path and/or the geometry of the internal walls of the lumen defining the flow path and/or the length of the flow path, and/or the presence or absence of constrictions in the flow path), the flow rate of gases exiting the gases exit port, the velocity of gases exiting the gases exit port and the resistance to flow through the gases exit port. The pressure differential may be determined by measuring the difference between pressure at the lumen second end and pressure within the connector body, or the difference between pressure at the lumen second end and atmosphere (as may be measured at or outside the gases exit port). Pressure measurements may be obtained using pressure sensors at these locations.

Furthermore, respiratory support provided by embodiments of the present disclosure causes little or no movement of the patient. This provides an opportunity for clinicians to undertake longer procedures on sedated or anaesthetised patients which require respiratory stillness, with less reliance on mechanical ventilation or other forms of respiratory support (such as e.g. high frequency jet ventilation (HFJV) or trans tracheal augmented ventilation (TTAV)) to ensure adequate respiratory support.

It is to be understood that various modifications, additions and/or alternatives may be made to the parts previously described without departing from the ambit of the present disclosure as defined in the claims appended hereto.

The disclosure may also be said broadly to consist in the parts, elements and features referred to or indicated in the specification of the application, individually or collectively, in any or all combinations of two or more of said parts, elements or features. Where, in the foregoing description reference has been made to integers or components having known equivalents thereof, those integers are herein incorporated as if individually set forth. Similarly, where in the foregoing description reference has been made to features or elements of a particular aspect or embodiment, it is to be understood that those features or elements are herein incorporated as if expressly disclosed in combination with other aspects or embodiments for which a skilled addressee would appreciate those features or elements to be compatible.

Where any or all of the terms “comprise”, “comprises”, “comprised” or “comprising” are used in this specification (including the claims) they are to be interpreted as specifying the presence of the stated features, integers, steps or components, but not precluding the presence of one or more other features, integers, steps or components or group thereof.

It is to be understood that the following claims are provided by way of example only, and are not intended to limit the scope of what may be claimed in future. Features may be added to or omitted from the claims at a later date so as to further define or re-define the invention or inventions.

The claims defining the invention are as follows:

1. A connector for coupling with an invasive patient interface providing respiratory support to a patient, the connector comprising:
(a) a connector body having:
(i) an inlet port couplable with a flow source providing a flow of respiratory gas;
(ii) a gases exit port; and
(iii) a device port couplable with the invasive patient interface; and
(b) a lumen having a first end and a second end;
wherein the connector body defines a gas flow path between the inlet port and both the device port and the lumen first end; and
wherein the lumen second end is disposed outside the device port.

2. The connector of claim 1, wherein the lumen second end is disposed outside the device port for delivery of gas at a target location deeper in the patient's airway than the device port.

3. The connector of claim 1, wherein the lumen provides an outflow path for gases entering the lumen second end at a target location deeper in the patient's airway than the device port.

4. The connector of claim 3, wherein the lumen first end is configured to provide for gases in the outflow path to flow to surrounding atmosphere.

5. The connector according to any one of the preceding claims, wherein the lumen second end is disposed, when in use, between the device port and a proximal end of the invasive patient interface.

6. The connector according to any one of the preceding claims, wherein the target location is within the invasive patient interface, close to a proximal end of the invasive patient interface.

7. The connector according to any one of the preceding claims, wherein at least part of the lumen is integral with the connector body.

8. The connector according to any one of the preceding claims, wherein the entire lumen is integral with the connector body.

9. The connector according to any one of claims 1 to 7, wherein the lumen comprises an elongate tube which, when in use, extends through at least part of the connector body.

10. The connector according to claim 9, wherein the connector body comprises a guide portion for positioning the elongate tube relative to the connector body.

11. The connector according to claim 10, wherein the guide portion is selected from a group comprising one or more of:
(a) a channel;
(b) an eyelet; and
(c) a groove or slot.

12. The connector according to claim 9 or claim 10, wherein the guide portion is disposed on an internal wall of the connector body defining a flow path from the inlet port to the device port.

13. The connector according to claim 9 or claim 10, wherein the guide portion is disposed on an internal wall of the connector body defining a flow path from the device port to the gases exit port.

14. The connector according to claim 9 or claim 10, wherein the guide portion is disposed externally of the connector body.

15. The connector according to any one of claims 9 to 14, wherein the guide portion is integral with the connector body.

16. The connector according to any one of the preceding claims comprising a locating feature for preventing the lumen from protruding out of the invasive patient interface when in use.

17. The connector according to claim 16, wherein the locating feature comprises an engagement structure adapted to couple with a cooperating structure on the invasive patient interface or an adapter connected to the invasive patient interface, the engagement structure and cooperating structure being respectively located to maintain a set back of the lumen second end within the invasive patient interface.

18. The connector according to any one of the preceding claims, configured for releasable coupling and recoupling with an invasive patient interface, either directly or through an adapter device.

19. The connector according to any one of the preceding claims, wherein the connector body defines a gas outflow path between the device port and the gases exit port.

20. The connector according to any one of the preceding claims, wherein the connector body defines a gas outflow path between the inlet port, the device port and the gases exit port.

21. The connector according to any one of the preceding claims, wherein the connector is configured to generate a predetermined airway pressure when coupled with the invasive patient interface in use.

22. The connector according to claim 21, wherein the connector includes one or more constrictions configured to generate the predetermined airway pressure.

23. The connector according to claim 22, wherein the one or more constrictions are disposed between the inlet port and the device port.

24. The connector according to claim 22 or claim 23, wherein the one or more constrictions are disposed upstream of where a gas flow path connecting the inlet port and the device port meets a gas outflow flow path connecting the device port and the gases exit port.

25. The connector according to any one of claims 21 to 24, wherein the predetermined pressure is at least about 2 cmH₂O and preferably in a range of about 2 cmH₂O to about 20 cmH₂O, preferably about 2 cmH₂O to about 10 cmH₂O, and more preferably about 2 cmH₂O to about 5 cmH₂O.

26. The connector according to any one of claims 21 to 25, wherein the flow of respiratory gas into the inlet port is divided between at least the device port and the lumen achieving a flow state in which airway pressure is maintained at the predetermined pressure.

27. The connector according to any one of the preceding claims, wherein the flow of respiratory gas into the inlet port is divided between the device port, the lumen and the gases exit port.

28. The connector according to any one of claims 1 to 26, wherein the flow of respiratory gas into the inlet port is divided only between the device port and the gases exit port achieving a flow state in which airway pressure is maintained at the predetermined pressure.

29. The connector according to any one of claims 1 to 27, wherein the flow of respiratory gas into the inlet port is divided between at least the device port and the lumen, achieving a flow state in which respiratory gas is delivered to the target location at a first predetermined velocity sufficient to achieve enhanced CO₂ clearance.

30. The connector according to claim 29, wherein the flow state achieves removal of gases from the target location at a second predetermined velocity of flow within the lumen sufficient to achieve enhanced CO₂ clearance.

31. The connector according to claim 29 or claim 30, wherein the lumen has dimensions configured to achieve one or both of the first and second predetermined velocity for gases exiting the lumen at the target location.

32. The connector according to any one of claims 29 to 31, wherein the lumen has an internal diameter which, relative to the internal diameter of the invasive patient interface, achieves one or both of the first and second predetermined velocity during use.

33. The connector according to claim 32, wherein the lumen has an internal diameter of about 2 mm to about 5 mm.

34. The connector according to any one of claims 29 to 33, wherein the lumen has an internal cross-sectional area which, relative to the internal cross-sectional area of the invasive patient interface, achieves one or both of the first and second predetermined velocity during use.

35. The connector according to any one of claims 27 and 30 to 34, wherein the predetermined velocity is in a range of about 5 m/s to about 25 m/s, preferably about 5 m/s to about 15 m/s m/s.

36. The connector according to any one of the preceding claims, wherein the inlet port receives a flow of respiratory gas having a flow rate in a range of about 10 LPM to about 150 LPM, preferably about 20 LPM to about 70 LPM.

37. The connector according to any one of the preceding claims, comprising a filter configured to treat gases in the outflow path.

38. The connector according to any one of the preceding claims, wherein the invasive patient interface is a sealing interface selected from a group comprising:
(a) an endotracheal tube;
(b) a laryngeal mask airway;
(c) a tracheostomy tube; and
(d) a suspension laryngoscope.

39. A system for providing respiratory support to a patient, the system comprising:
(a) a flow source providing a flow of respiratory gas,
(b) the connector according to any one of the preceding claims; and
(c) an invasive patient interface.

40. The system according to claim 39, wherein the flow source provides flows of respiratory gas at a flow rate in a range of about 10 LPM to about 120 LPM, preferably about 20 LPM to about 70 LPM.
